(19) United States
(12) Patent Application Publication
(10) Pub. No.: US 2015/0057216 A1
(43) Pub. Date: Feb. 26, 2015

(54) ANGIOTENSINS FOR TREATMENT OF FIBROSIS

(71) Applicant: TARIX PHARMACEUTICALS LTD., Cambridge, MA (US)

(72) Inventors: Paul Beringer, Arcadia, CA (US); Richard Franklin, Cambridge, MA (US)

(21) Appl. No.: 14/365,565

(22) PCT Filed: Dec. 14, 2012
(86) PCT No.: PCT/US12/69930

§ 371 Date: Jun. 13, 2014

Related U.S. Application Data
(60) Provisional application No. 61/576,673, filed on Dec. 16, 2011.
(60) Provisional application No. 61/579,936, filed on Dec. 23, 2011.

(51) Int. Cl. A61K 38/08

(52) U.S. Cl. CPC A61K 38/085
USPC 514/1.7; 514/9.7; 514/1.8

(57) ABSTRACT

The present invention provides, among other things, methods and compositions for treating or preventing fibrotic diseases, disorders or conditions based on Angiotensin (1-7) polypeptides, and analogs or derivatives thereof. In some embodiments, compositions and methods for treating or preventing pulmonary fibrosis, pulmonary hypertension, chronic obstructive pulmonary disease (COPD), asthma, cystic fibrosis, kidney fibrosis, liver fibrosis, systemic sclerosis, post-surgical adhesions, accelerating wound healing, and reducing or preventing scar formation are provided.

### CROSS REFERENCE TO RELATED APPLICATIONS

This application claims priority from U.S. Provisional Application Ser. No. 61/576,673, filed on Dec. 16, 2011 and from U.S. Provisional Application Ser. No. 61/579,936, filed Dec. 23, 2011, the disclosures of which are hereby incorporated by reference in their entirety.

### BACKGROUND

Fibrosis is characterized by the formation of excessive fibrous tissue, such as connective tissue. Fibrosis can result from acute or chronic injury or disease. Fibrosis often results in irreversible tissue damage that can severely impair organ function. Many fibrotic disorders do not respond to current treatment; thus, new treatments are needed that can prevent or reduce fibrosis.

Cystic fibrosis is a subtype of fibrosis that is characterized by a chronic cycle of airway obstruction, infection, and inflammation, leading to remodeling, impaired pulmonary function, and eventual respiratory failure. The chronic inflammatory response in the airways is mediated by overexuberant neutrophil infiltration in response to chemokines (e.g. CXCL8) resulting in release of excessive proteases (e.g. neutrophil elastase, matrix metalloproteinase-9). Lung biopsies show inflammatory cell infiltration and loss of bronchiolar epithelial cells with increased reticular basement membrane thickness indicative of airway remodeling. Pooled data from 4 multicenter trials demonstrated a significant correlation between pulmonary disease severity and sputum neutrophils and concentrations of neutrophil elastase from patients with CF. Such data demonstrates the significant role of chronic airway inflammation in the pathophysiology of lung disease in CF and underscores the importance of therapeutic intervention to preserve pulmonary function.

### SUMMARY

The invention provides, among other things, compositions and methods for improved and more efficient treatment and/or prevention of fibrosis and various fibrotic diseases, disorders or conditions. As described in the Examples section below, the present invention is, in part, based on the surprising discovery that the use of Angiotensin (1-7) or analogs or derivatives thereof, including both linear and cyclic angiotensin peptides, may result in a decrease in fibrotic symptoms including collagen deposition, inflammatory cell infiltration and decreased expression of pro-fibrotic genes including Collagen Type 3 and α-SMA.

It is contemplated that the present invention can be used to treat various fibrotic diseases, disorders, and conditions, including but not limited to systemic sclerosis, multifocal fibrosclerosis, sclerodermatous graft-vs-host-disease, nephrogenic systemic fibrosis, organ specific fibrosis, and the like.

In some embodiments, the present invention provides a method of treating or preventing a fibrotic disease, disorder or condition by administering to a subject in need of treatment Angiotensin (1-7), an analog or derivative thereof.

In some embodiments, the present invention can be used to treat or prevent lung fibrosis. In some embodiments, the lung fibrosis that can be treated by the present invention is selected from the group consisting of pulmonary fibrosis, pulmonary hypertension, chronic obstructive pulmonary disease (COPD), asthma, cystic fibrosis, and combinations thereof. In particular embodiments, the present invention can be used to treat or prevent cystic fibrosis.

In some embodiments, the present invention can be used to treat or prevent kidney fibrosis.

In some embodiments, the present invention can be used to treat or prevent liver fibrosis, such as, nonalcoholic steatohepatitis (NASH).

In some embodiments, the present invention can be used to treat or prevent heart fibrosis, for example, endomyocardial fibrosis.

In some embodiments, the present invention can be used to treat or prevent systemic sclerosis.

In some embodiments, the present invention can be used to treat or prevent fibrotic diseases, disorders or conditions caused by post-surgical adhesion formation.

In some embodiments, the present invention provides a method for accelerating wound healing in a subject by administering to a subject in need of treatment Angiotensin (1-7) or an analog or derivative thereof.

In some embodiments, the present invention provides a method for reducing or preventing scar formation in a subject by administering to a subject in need of treatment Angiotensin (1-7) or an analog or derivative thereof. In some embodiments, the present invention can be used to reduce or prevent scar formation on skin.

In various embodiments, an Angiotensin (1-7) or analog or derivative thereof is administered at a therapeutically effective amount such that at least one symptom or feature of a fibrotic disease, disorder or condition, or other related diseases, disorders or conditions, is reduced in intensity, severity, or frequency, or has delayed onset.

It is contemplated that various embodiments may use different amounts of an Angiotensin (1-7) or analog or derivative thereof. In some embodiments, an Angiotensin (1-7) or analog or derivative thereof is administered at an effective dose ranging from about 0.0001-1,000 mg/kg/day (e.g., about 0.001-100 mg/kg/day, about 0.001-10 mg/kg/day, about 0.001-1 mg/kg/day, about 1-900 μg/kg/day, about 1-800 μg/kg/day, about 1-700 μg/kg/day, about 1-600 μg/kg/day, about 1-500 μg/kg/day, about 1-400 μg/kg/day, about 1-300 μg/kg/day, about 1-200 μg/kg/day, about 1-100 μg/kg/day, about 1-90 μg/kg/day, about 1-80 μg/kg/day, about 1-70 μg/kg/day, about 1-60 μg/kg/day, about 1-50 μg/kg/day, about 1-40 μg/kg/day, about 1-30 μg/kg/day, about 1-20 μg/kg/day, about 1-10 μg/kg/day). In some embodiments, an Angiotensin (1-7) or analog or derivative thereof is administered at an effective dose selected from about 1, 2, 4, 6, 8, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, or 1,000 μg/kg/day.

As used herein, an Angiotensin (1-7) or analog or derivative thereof includes naturally-occurring Angiotensin (1-7), a functional equivalent thereof, and any Angiotensin (1-7) agonist including Angiotensin (1-7) receptor agonist. As used herein, a functional equivalent of naturally-occurring Angiotensin (1-7) refers to any peptide that shares amino acid sequence identity to the naturally-occurring Angiotensin (1-7) and retain substantially the same or similar activity as the naturally-occurring Angiotensin (1-7). As used herein, the term “angiotensin-(1-7) receptor’ encompasses the G Protein-Coupled Mas Receptors. As used herein, “Angiotensin (1-7) agonist” or “Angiotensin-(1-7) receptor agonist” encompasses any molecule that has a positive impact in a function of Angiotensin-(1-7) or an angiotensin-(1-7) receptor, in particular, the G-protein coupled Mas receptor. For example, an Angiotensin (1-7) or Angiotensin-(1-7) receptor agonist directly or indirectly enhances, strengthens, activates and/or increases Angiotensin (1-7) or an angiotensin-(1-7) receptor (i.e., the Mas receptor) activity. In some embodiments, an angiotensin-(1-7) receptor agonist directly interacts with an angiotensin-(1-7) receptor (i.e., the Mas receptor). Such agonists can be peptidic or non-peptidic including, e.g., proteins, chemical compounds, small molecules, nucleic acids, antibodies, drugs, ligands, or other agents.

In various embodiments, an Angiotensin (1-7) or analog or derivative thereof suitable for the present invention is naturally-occurring Angiotensin (1-7) with amino acid sequence of Asp¹-Arg²-Val³-Tyr⁴-Ile⁵-His⁶-Pro⁷ (SEQ ID NO: 1).

In various embodiments, an Angiotensin (1-7) or analog or derivative thereof suitable for the invention is a functional equivalent of naturally-occurring Angiotensin (1-7) having amino acid sequence of Asp¹-Arg²-Nle³-Tyr⁴-Ile⁵-His⁶-Pro⁷ (SEQ ID NO: 2).

In various embodiments, an Angiotensin (1-7) or analog or derivative thereof suitable for the invention is a functional equivalent of naturally-occurring Angiotensin (1-7) having amino acid sequence of Asp¹-Arg²-Val³-Ser⁴-Ile⁵-His⁶-Cys⁷ (SEQ ID NO: 3).

In various embodiments, an Angiotensin (1-7) or analog or derivative thereof suitable for the invention is a functional equivalent of naturally-occurring Angiotensin (1-7) that is a cyclic Angiotensin (1-7) polypeptide. In some embodiments, the cyclic peptide comprises a linkage between amino acids. In some embodiments, the linkage is located at residues corresponding to positions Tyr⁴ and Pro⁷ in naturally-occurring Angiotensin (1-7). In some embodiments, the linkage is a thioether bridge. In some embodiments, the cyclic peptide comprises an amino acid sequence otherwise identical to the naturally-occurring Angiotensin (1-7) amino acid sequence of Asp¹-Arg²-Val³-Tyr⁴-Ile⁵-His⁶-Pro⁷ (SEQ ID NO:1). In some embodiments, the cyclic peptide comprises a norleucine (Nle) replacing position Val3 in naturally-occurring Angiotensin (1-7). In some embodiments, the cyclic peptide is a 4,7-cyclized angiotensin (1-7) with the following formula Asp¹-Arg²-Val³-Ser⁴-Ile⁵-His⁶-Cys⁷ (SEQ ID NO: 3). In particular embodiments, a suitable cyclic Angiotensin (1-7) polypeptide is a 4,7-cyclized Angiotensin (1-7) with the following formula:

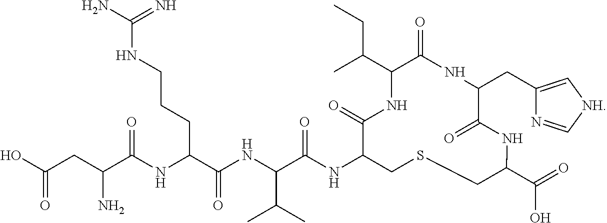 [SMILES: CCC(C)C1NC(=O)C(NC(=O)C(NC(=O)C(CCCNC(=N)N)NC(=O)C(N)CC(=O)O)C(C)C)CSCC(C(=O)O)NC(=O)C(Cc2c[nH]cn2)NC1=O]

In some embodiments, the Angiotensin (1-7) or an analog or derivative thereof comprises one or more chemical modifications to increase protease resistance, serum stability and/or bioavailability. In some embodiments, the one or more chemical modifications comprise pegylation.

In some embodiments, the Angiotensin (1-7) or an analog or derivative thereof is Angiotensin (1-7) receptor agonist. In some embodiments, an Angiotensin (1-7) receptor agonist suitable for the invention is a non-peptidic agonist. In some embodiments, an Angiotensin (1-7) receptor agonist is a 1-(p-thienylbenzyl)imidazole. In some embodiments, the 1-(p-thienylbenzyl)imidazole has the following formula:

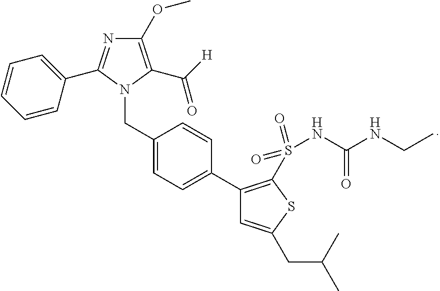 [SMILES: CCNC(=O)NS(=O)(=O)c1sc(CC(C)C)cc1-c1ccc(Cn2c(-c3ccccc3)nc(OC)c2C=O)cc1]

In various embodiments, an Angiotensin (1-7) or analog or derivative thereof is administered parenterally. For example, suitable parenteral administration can be intravenous, intradermal, inhalation, transdermal (topical), subcutaneous, and/or transmucosal administration.

In various embodiments, an Angiotensin (1-7) or analog or derivative thereof is administered orally.

In some embodiments, an Angiotensin (1-7) or analog or derivative thereof is administered bimonthly, monthly, triweekly, biweekly, weekly, daily, or at variable intervals.

As used in this application, the terms “about” and “approximately” are used as equivalents. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

The drawings are for illustration purposes only not for limitation.

FIG. 1 shows graphs displaying exemplary measured levels of: A) whole blood glucose, B) plasma alanine transaminase (ALT), C) plasma aspartate transaminase (AST), and D) plasma alkaline phosphatase (ALP) in mice receiving no treatment, saline, Telmisartan, or 30, 100, 300 or 1,000 μg/kg of TXA127.

FIG. 2 shows a graph displaying hydroxyproline levels in the liver of mice receiving no treatment, saline, Telmisartan, or 30, 100, 300 or 1,000 μg/kg of TXA127.

FIG. 3 shows a graph displaying the percentages of Sirius-positive area in the livers of mice receiving no treatment, saline, Telmisartan, or 30, 100, 300 or 1,000 μg/kg of TXA127.

FIG. 4 shows a graph displaying the mRNA expression levels of: A) Collagen Type I, B) Collagen Type 3, C) α-SMA, and D) TGF-β in the liver of mice receiving no treatment, saline, Telmisartan, or 30, 100, 300 or 1,000 μg/kg of TXA127.

FIG. 5 shows a graph displaying the mRNA expression levels of: A) CCR2, and B) TIMP-1 in the liver of mice receiving no treatment, saline, Telmisartan, or 30, 100, 300 or 1,000 μg/kg of TXA127.

### DEFINITIONS

In order for the present invention to be more readily understood, certain terms are first defined. Additional definitions for the following terms and other terms are set forth throughout the specification.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). As used in this application, the terms “about” and “approximately” are used as equivalents.

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion. In certain embodiments, a peptide has no intrinsic biological activity but that inhibits the effects of one or more naturally-occurring angiotensin compounds is considered to be biologically active.

Carrier or diluent: As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g. phosphate-buffered saline), sterile saline solution, Ringer's solution or dextrose solution.

Comprise: As used herein, the term “comprise” and variations of the term, such as “comprising” and “comprises,” are not intended to exclude other additives, components, integers or steps.

Dosage form: As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses. In some embodiments, the therapeutic agent is administered continuously over a predetermined period. In some embodiments, the therapeutic agent is administered once a day (QD) or twice a day (BID).

Dysfunction: As used herein, the term “dysfunction” refers to an abnormal function. Dysfunction of a molecule (e.g., a protein) can be caused by an increase or decrease of an activity associated with such molecule. Dysfunction of a molecule can be caused by defects associated with the molecule itself or other molecules that directly or indirectly interact with or regulate the molecule.

Functional equivalent or derivative: As used herein, the term “functional equivalent” or “functional derivative” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subjects) in the absence of the treatment described herein. A “control subject” is a subject afflicted with the same form of disease as the subject being treated, who is about the same age as the subject being treated.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

Prevent: As used herein, the term “prevent” or “prevention”, when used in connection with the occurrence of a disease, disorder, and/or condition, refers to reducing the risk of developing the disease, disorder and/or condition. See the definition of “risk.”

Polypeptide: The term “polypeptide” or “peptide” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Risk: As will be understood from context, a “risk” of a disease, disorder, and/or condition comprises a likelihood that a particular individual will develop a disease, disorder, and/or condition (e.g., Fibrosis). In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event (e.g., Diabetes). In some embodiments a reference sample or group of reference samples are from individuals comparable to a particular individual. In some embodiments, relative risk is 0, 1, 2, 3, 4, 5, 6, 7, 8, 9, 10, or more.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, condition, or event (for example, ischemic stroke) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein associated with the disease, disorder, and/or condition; (4) habits and/or lifestyles associated with development of the disease, disorder, condition, and/or event (5) having undergone, planning to undergo, or requiring a transplant. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition. A subject susceptible to a fibrotic disorder also includes a subject that has received an injury or has undergone or is about to undergo a surgical procedure.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease. Treatment may be administered to prevent a fibrotic disorder in a subject susceptible to a fibrotic disorder.

### DETAILED DESCRIPTION

Described herein are, among other things, methods for treating fibrotic or other related diseases, disorders or conditions. In some embodiments, inventive methods according to the present invention comprise administering to a subject in need Angiotensin (1-7), analog or derivative thereof, or a pharmaceutical composition containing the same. In particular, an Angiotensin (1-7), analog or derivative thereof is administered in a therapeutically effective amount such that at least one symptom or feature of a fibrotic disease, disorder or condition, or other related diseases, disorders or conditions, is reduced in intensity, severity, or frequency, or has delayed onset.

Various aspects of the invention are described in detail in the following sections. The use of sections is not meant to limit the invention. Each section can apply to any aspect of the invention. In this application, the use of “or” means “and/or” unless stated otherwise.

### Fibrotic Disorders and Conditions

As used herein, the term “fibrotic disorder” refers to any disease characterized by fibrosis, including but not limited to systemic sclerosis, multifocal fibrosclerosis, sclerodermatous graft-vs-host-disease, nephrogenic systemic fibrosis, organ specific fibrosis, and the like. Illustrative organ specific fibrotic disorders include, but are not limited to, pulmonary fibrosis, pulmonary hypertension, cystic fibrosis, asthma, chronic obstructive pulmonary disease, liver fibrosis, kidney fibrosis, NASH, and the like. Many fibrotic diseases, disorders or conditions have disordered and/or exaggerated deposition of extracellular matrix in affected tissues. Fibrosis may be associated with inflammation, occur as a symptom of underlying disease, and/or caused by surgical procedure or wound healing process. Unchecked fibrosis can result in destruction of the architecture of the underlying organ or tissue, commonly referred to as scarring.

The present invention provides methods of treating various fibrotic diseases, disorders, or conditions as described in greater detail below.

For example, fibrosis of the lungs represents a debilitating and potentially fatal form of fibrosis. Treatment options for fibrosis in lung tissue are very limited. Once developed, scarring is permanent, and lung transplantation is often the only therapeutic option available.

Pulmonary fibrosis is characterized by progressive scarring of lung tissue accompanied by fibroblast proliferation, excessive accumulation of extracellular matrix proteins, and abnormal alveolar structure. The thickened and stiff tissue makes it difficult for lungs to work properly, leading to breathing problems such as shortness of breath, and can ultimately be fatal. Pulmonary fibrosis may be caused by acute lung injury, viral infection, exposure to toxins, radiation, chronic disease, medications, or may be idiopathic (i.e., an undiscovered underlying cause).

The classic findings in idiopathic pulmonary fibrosis show diffuse peripheral scarring of the lungs with small bubbles (known as bullae) adjacent to the outer lining of the surface of the lung, often at the bases of the lungs. Idiopathic pulmonary fibrosis often has a slow and relentless progression. Early on, patients often complain of a dry unexplained cough. Next, shortness of breath (dyspnea) sets in and worsens over time triggered by less and less activity. Eventually, the shortness of breath becomes disabling, limiting all activity and even occurring while sitting still. In rarer cases, the fibrosis can be rapidly progressive, with dyspnea and disability occurring in weeks to months of onset of the disease. This form of pulmonary fibrosis has been referred to as Hamman-Rich syndrome.

Pulmonary hypertension is marked by an increase in the blood pressure of the lung vasculature, including the pulmonary artery, pulmonary vein, and/or pulmonary capillaries. Abnormally high pressure strains the right ventricle of the heart, causing it to expand. Over time, the right ventricle can weaken and lose its ability to pump enough blood to the lungs, leading to the development of heart failure. Pulmonary hypertension can occur as a result of other medical conditions, such as chronic liver disease and liver cirrhosis; rheumatic disorders such as scleroderma or systemic lupus erythematosus (lupus); and lung conditions including tumors, emphysema, chronic obstructive pulmonary disease (COPD), and pulmonary fibrosis. Pulmonary fibrosis may lead to narrowing of pulmonary vasculature resulting in pulmonary hypertension.

Chronic Obstructive Pulmonary Disease (COPD) is a common lung disease that is often associated with chronic bronchitis or emphysema. Symptoms can often include cough, mucus build up, fatigue, wheezing, and respiratory infection.

Chronic bronchitis and emphysema are diseases of the lungs in which the airways become narrowed. This leads to a limitation of the flow of air to and from the lungs, causing shortness of breath (dyspnea). In clinical practice, COPD is defined by its characteristically low airflow on lung function tests.

Lung damage and inflammation in the large airways results in chronic bronchitis. In the airways of the lung, the hallmark of chronic bronchitis is an increased number (hyperplasia) and increased size (hypertrophy) of the goblet cells and mucous glands of the airway. As a result, there is more mucus than usual in the airways, contributing to narrowing of the airways and causing a cough with sputum. Microscopically there is infiltration of the airway walls with inflammatory cells. Inflammation is followed by scarring and remodeling that thickens the walls and also results in narrowing of the airways. As chronic bronchitis progresses, there is squamous metaplasia (an abnormal change in the tissue lining the inside of the airway) and fibrosis (further thickening and scarring of the airway wall). The consequence of these changes is a limitation of airflow and difficulty breathing.

Asthma is a chronic lung disease characterized by inflammation and constriction of the airways. Asthma causes recurring periods of wheezing, tightness of the chest, shortness of breath, and coughing. Swelling and overproduction of mucus can cause further airway constriction and worsening of symptoms. There is evidence that increased matrix degradation may occur in asthma, and this may contribute to mechanical changes in the airways in asthma (Roberts et al (1995) Chest 107:111 S-117S, incorporated herein by reference in its entirety. Treatment of extracellular matrix degradation may ameliorate symptoms of asthma.

Cystic fibrosis is a recessive multi-system genetic disease characterized by abnormal transport of chloride and sodium across epithelium, leading to thick, viscous secretions in the lungs, pancreas, liver, intestine and reproductive tract. Cystic fibrosis is caused by a mutation in the gene for the protein cystic fibrosis transmembrane conductance regulator (CFTR). Lung disease results from clogging of the airways due to mucus build-up, decreased mucociliary clearance, and resulting inflammation, which can cause fibrotic injury and structural changes to the lungs. The fibrotic lung damage progresses over time leading some cystic fibrosis patients to require lung transplant.

When referring to cystic fibrosis, to “treat” or “treating” cystic fibrosis (CF) may mean accomplishing one or more of the following: (a) reducing inflammation in the subject; (b) limiting any increase in inflammation the subject; (c) reducing the severity of one or more CF symptoms; (d) limiting or preventing development of one or more CF symptoms; (e) inhibiting worsening of one or more CF symptoms; and (f) limiting or preventing recurrence of one or more CF symptoms in subjects that were previously symptomatic for the relevant CF symptom.

Common symptoms of subjects suffering from cystic fibrosis include, but are not limited to, accumulation of thick mucus, copious phlegm production, frequent chest infections, frequent coughing, frequent shortness of breath, inflammation, decreased ability to exercise, opportunistic infections of the lung and sinus (including but not limited to Staphylococcus aureus, Haemophilus influenzae, Mycobacterium aviium, and Pseudomonas aeruginosa), pneumonia, tuberculosis, bronchiectasis, hemoptysis, pulmonary hypertension (and resulting heart failure), hypoxia, respiratory failure, allergic bronchopulmonary aspergillosis, mucus in the paranasal sinuses, sinus infection, facial pain, fever, excessive nasal drainage, development of nasal polyps, cardiorespiratory complications, CF-related diabetes, rectal prolapse, pancreatitis, malabsorption, intestinal blockage, exocrine pancreatic insufficiency, bile duct blockage, and liver cirrhosis.

In some embodiments, the symptoms of cystic fibrosis comprise inflammation. In these embodiments, some methods within the scope of the invention may comprise treating inflammation, wherein a beneficial effect of treatment can be assessed by, for example, a reduction in inflammatory cell count in a relevant sample from the subject, such as bronchoalveolar lavage (BAL) fluid. In a non-limiting embodiment, the beneficial effect may be assessed by demonstrating a reduction in neutrophil count in BAL fluid from the subject. The excessive recruitment of neutrophils into the airways of patients with CF is a significant predictor of lung disease severity in CF and therefore is an important therapeutic target. Methods for measuring such cell counts are well known in the art, including but not limited to FACS techniques. Thus, in some embodiments, methods within the scope of the invention may comprise reducing inflammation in the subject. In some embodiments, the method may comprise reducing neutrophil cell count in BAL fluid from the subject compared to control. Any suitable control can be used for comparison, such as cystic fibrosis subjects not treated with the peptide. In some embodiments, a decrease in neutrophil count provides a clinical benefit to the subject. In various embodiments, the reduction in neutrophil count is at least 5%, 10%, 15%, 20%, 25%, 50%, or more compared to control.

In another embodiment, the beneficial effect of the therapeutic methods of the invention may be assessed by a reduction in one or more inflammatory biomarkers in a relevant sample from the subject, such as BAL fluid. In various non-limiting embodiments, the inflammatory biomarker may comprise or consist of one or more of IL1β, KC, MIP2, IFNγ, TNFα, IL-6, MCP-1, and IL-10 in BAL fluid. Methods for measuring the amount of such biomarkers are well known in the art, including but not limited to ELISAs. Thus, in this embodiment, the methods may further comprise the reducing an amount of one or more inflammatory biomarkers in a BAL sample from the subject compared to control.

Post-surgical adhesion formation is a common complication of surgery. The formation of adhesions, from mechanical damage, ischemia, and infections, can increase morbidity and mortality following surgery. Although refined surgical procedures can reduce the magnitude of adhesion formation, adhesions are rarely eviscerated and an effective adjunctive therapy is needed. Reducing the fibrosis associated with this process could reduce pain, obstruction and other complications of surgery and promote healing and recovery.

Wounds (i.e., lacerations, openings) in mammalian tissue result in tissue disruption and coagulation of the microvasculature at the wound face. Repair of such tissue represents an orderly, controlled cellular response to injury. Soft tissue wounds, regardless of size, heal in a similar manner. Tissue growth and repair are biologic systems wherein cellular proliferation and angiogenesis occur in the presence of an oxygen gradient. The sequential morphological and structural changes which occur during tissue repair have been characterized in detail and have in some instances been quantified (see e.g., Hunt, T. K., et al., “Coagulation and macrophage stimulation of angiogenesis and wound healing,” in The Surgical Wound, pp. 1-18, ed. F. Dineen & G. Hildrick-Smith (Lea & Febiger, Philadelphia: 1981)). The cellular morphology consists of three distinct zones. The central avascular wound space is oxygen deficient, acidotic and hypercarbic, and has high lactate levels. Adjacent to the wound space is a gradient zone of local anemia (ischemia) which is populated by dividing fibroblasts. Behind the leading zone is an area of active collagen synthesis characterized by mature fibroblasts and numerous newly-formed capillaries (i.e., neovascularization). U.S. Pat. Nos. 5,015,629 and 7,022,675 (each incorporated by reference herein) disclose methods and compositions for increasing the rate of wound repair.

Scar formation is a natural part of the healing process. Disorderly collagen synthesis and deposition in a wound can result in excessive, thick, or raised scar formation. Generally, the larger the wound, the longer it takes to heal and the greater the chance of a problematic scar.

There are several types of scars. Hypertropic scars are raised, pinkish-red areas located inside the borders of the original injury. They are often described as itchy. In some cases, hypertropic scars shrink and fade on their own. Keloids are raised, deep-red areas that tend to cover much more area than that of the original injury. Even when surgically removed, keloids tend to recur. Atrophic scars are skin depressions, like those that sometimes form from severe acne. They are caused by inflammation that destroys the collagen during the rebuilding process, leaving an area of indentation.

Systemic sclerosis is a systemic connective tissue disease characterized by alterations of the microvasculature, disturbances of the immune system and by massive deposition of collagen and other matrix substances in the connective tissue. Systemic sclerosis is a clinically heterogeneous generalized disorder which affects the connective tissue of the skin and internal organs such as gastrointestinal tract, lungs, heart and kidneys. Reduction of fibrosis resulting from systemic sclerosis may ameliorate symptoms and/or prevent further complications in affected tissues.

Nonalcoholic steatohepatitis (NASH) is a common liver disease. It resembles alcoholic liver disease but occurs in people who drink little or no alcohol. The major feature in NASH is fat in the liver, along with inflammation and damage. Nevertheless, NASH can be severe and can lead to cirrhosis, in which the liver is permanently damaged and scarred and no longer able to work properly.

NASH is usually a silent disease with few or no symptoms. Patients generally feel well in the early stages and only begin to have symptoms—such as fatigue, weight loss, and weakness—once the disease is more advanced or cirrhosis develops. The progression of NASH can take years, even decades. The process can stop and, in some cases may even begin to reverse on its own without specific therapy. Or NASH can slowly worsen, causing scarring or fibrosis to appear and accumulate in the liver. As fibrosis worsens, cirrhosis develops in which the liver becomes seriously scarred, hardened, and unable to function normally. Not every person with NASH develops cirrhosis, but once serious scarring or cirrhosis is present, few treatments can halt the progression. A person with cirrhosis experiences fluid retention, muscle wasting, bleeding from the intestines, and liver failure. Liver transplantation is the only treatment for advanced cirrhosis with liver failure, and transplantation is increasingly performed in people with NASH. NASH ranks as one of the major causes of cirrhosis in America, behind hepatitis C and alcoholic liver disease.

Kidney (renal) fibrosis results from excessive formation of fibrous connective tissue in the kidney. Kidney fibrosis causes significant morbidity and mortality and leads to a need for dialysis or kidney transplantation. Fibrosis can occur in either the filtering or reabsorptive component of the nephron, the functional unit of the kidney. A number of factors may contribute to kidney scarring, particularly derangements of physiology involved in the autoregulation of glomerular filtration. This in turn leads to replacement of normal structures with accumulated extracellular matrix. A spectrum of changes in the physiology of individual cells leads to the production of numerous peptide and non-peptide fibrogens that stimulate alterations in the balance between extracellular matrix synthesis and degradation to favor scarring.

### Angiotensin (1-7) and Analogs or Derivatives Thereof

Angiotensins are polypeptides of the renin-angiotensin system. The circulating renin-angiotensin system (RAS) has a well-described role in circulatory homeostasis. Local tissue-based RAS also exist, such as in lung, and play a role in injury and repair responses. Naturally-occurring Angiotensin (1-7) is a linear polypeptide having an amino acid sequence of Asp¹-Arg²-Val³-Tyr⁴-Ile⁵-His⁶-Pro⁷ (SEQ ID NO: 1). In the renin-angiotensin system, the vasodilating activity of angiotensin-(1-7) counteracts the vasoconstricting activity of angiotensin II. Ang-(1-7) is an endogenous ligand for Mas receptors. Mas receptors are G-protein coupled receptors containing seven transmembrane spanning regions. Without wishing to be bound by theory, it is hypothesized that administration of an Angiotensin (1-7) polypeptide or an analog and derivative thereof may exert anti-fibrotic effects via activation of Mas receptors.

An Angiotensin (1-7) or analog or derivative thereof suitable for the present invention includes naturally-occurring Angiotensin (1-7), a functional equivalent thereof, and any Angiotensin (1-7) agonist including Angiotensin (1-7) receptor agonist. As used herein, a functional equivalent of naturally-occurring Angiotensin (1-7) refers to any peptide that shares amino acid sequence identity to the naturally-occurring Angiotensin (1-7) and retain substantially the same or similar activity as the naturally-occurring Angiotensin (1-7). The terms “peptide” and “polypeptide” are used interchangeably in this application. As used herein, the term “angiotensin-(1-7) receptor’ encompasses the G Protein-Coupled Mas Receptors. As used herein, “Angiotensin (1-7) agonist” or “Angiotensin-(1-7) receptor agonist” encompasses any molecule that has a positive impact in a function of Angiotensin-(1-7) or an angiotensin-(1-7) receptor, in particular, the G-protein coupled Mas receptor. For example, an Angiotensin (1-7) or Angiotensin-(1-7) receptor agonist directly or indirectly enhances, strengthens, activates and/or increases Angiotensin (1-7) or an angiotensin-(1-7) receptor (i.e., the Mas receptor) activity. In some embodiments, an angiotensin-(1-7) receptor agonist directly interacts with an angiotensin-(1-7) receptor (i.e., the Mas receptor). Such agonists can be peptidic or non-peptidic including, e.g., proteins, chemical compounds, small molecules, nucleic acids, antibodies, drugs, ligands, or other agents.

In certain embodiments, compositions comprising Ang(1-7) polypeptides contain additional amino acids linked to an Ang(1-7) polypeptide so as to contain more than seven contiguous amino acids. In certain embodiments, compositions comprising Ang(1-7) polypeptides contain one or more amino acids deleted from an Ang(1-7) polypeptide so as to contain fewer than 7 contiguous amino acids.

In certain embodiments, compositions comprising an Ang(1-7) polypeptide contain one or more modifications made to the Ang(1-7) polypeptide to increase protease resistance, serum stability and/or bioavailability. In some embodiments, suitable modifications are selected from acetylation, glycosylation, biotinylation, pegylation, substitution with D-amino acid and/or un-natural amino acid, and/or cyclization of the peptide.

Ang(1-7) polypeptide derivatives can be made by altering the amino acid sequences by substitution, addition, or deletion or an amino acid residue to provide a functionally equivalent molecule, or functionally enhanced or diminished molecule, as desired. The derivatives of the present invention include, but are not limited to, those containing, as primary amino acid sequence, all or part of the amino acid sequence of SEQ ID NO: 1, including altered sequences containing substitutions of functionally equivalent amino acid residues. For example, one or more amino acid residues within the sequence can be substituted by another amino acid of a similar polarity, which acts as a functional equivalent, resulting in a silent alteration. Substitution for an amino acid within the sequence may be selected from other members of the class to which the amino acid belongs. For example, the positively charged (basic) amino acids include arginine, lysine, and histidine. The nonpolar (hydrophobic) amino acids include leucine, isoleucine, alanine, phenylalanine, valine, proline, tryptophane, and methionine. The uncharged polar amino acids include serine, threonine, cysteine, tyrosine, asparagine, and glutamine. The negatively charged (acid) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions. For example, the amino acid sequence of a peptide inhibitor can be modified or substituted.

As used herein, the term “amino acid,” in its broadest sense, refers to any compound and/or substance that can be incorporated into a polypeptide chain. In certain embodiments, an amino acid has the general structure H₂N—C(H)(R)—COOH. In certain embodiments, an amino acid is a naturally-occurring amino acid. In certain embodiments, an amino acid is a synthetic or un-natural amino acid (e.g., α,α-disubstituted amino acids, N-alkyl amino acids); in some embodiments, an amino acid is a D-amino acid; in certain embodiments, an amino acid is an L-amino acid, a combination of D- and L-amino acids, and/or various “designer” amino acids (e.g., β-methyl amino acids, Cα-methyl amino acids, and Nα-methyl amino acids, etc.) to convey special properties. In some embodiments, the N-terminus may be acetylated and/or the C-terminus may be amidated. “Standard amino acid” refers to any of the twenty standard amino acids commonly found in naturally occurring peptides including both L- and D-amino acids which are both incorporated in peptides in nature. “Nonstandard” or “unconventional amino acid” refers to any amino acid, other than the standard amino acids, regardless of whether it is prepared synthetically or obtained from a natural source. As used herein, “synthetic or un-natural amino acid” encompasses chemically modified amino acids, including but not limited to salts, amino acid derivatives (such as amides), and/or substitutions.

Amino acids, including carboxy- and/or amino-terminal amino acids in peptides, can be modified by methylation, amidation, acetylation, and/or substitution with other chemical groups that can change the peptide's circulating half-life without adversely affecting its activity. Examples of unconventional or un-natural amino acids include, but are not limited to, citrulline, ornithine, norleucine, norvaline, 4-(E)-butenyl-4(R)-methyl-N-methylthreonine (MeBmt), N-methyl-leucine (MeLeu), aminoisobutyric acid, statine, and N-methyl-alanine (MeAla). Amino acids may participate in a disulfide bond. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

In certain embodiments, the Ang(1-7) polypeptides contain one or more L-amino acids, D-amino acids, and/or un-natural amino acids.

As used herein, the term “reverse-D peptide” refers to peptides containing D-amino acids, arranged in a reverse sequence relative to a peptide containing L-amino acids. For example, the C-terminal residue of an L-amino acid peptide becomes N-terminal for the D-amino acid peptide, and so forth. Reverse D-peptides desirably retain the same tertiary conformation and therefore the same activity, as the L-amino acid peptides, but desirably are more stable to enzymatic degradation in vitro and in vivo, and therefore can have greater therapeutic efficacy than the original peptide (Brady and Dodson, Nature 368:692-693, 1994; and Jameson and McDonnel, Nature 368:744-746, 1994).

As used herein, the term “reverse-L peptide” refers to peptides containing L-amino acids arranged in a reverse sequence relative to a parent peptide. The C-terminal residue of the parent peptide becomes N-terminal for the reverse-L peptide, and so forth.

In addition to peptides containing only naturally occurring amino acids, peptidomimetics or peptide analogs are also encompassed by the present invention. Peptide analogs are commonly used in the pharmaceutical industry as non-peptide drugs with properties analogous to those of the template peptide. The non-peptide compounds are termed “peptide mimetics” or peptidomimetics (Fauchere et al., Infect. Immun. 54:283-287 (1986); Evans et al., J. Med. Chem. 30:1229-1239 (1987)). Peptide mimetics that are structurally related to therapeutically useful peptides may be used to produce an equivalent or enhanced therapeutic or prophylactic effect. Generally, peptidomimetics are structurally similar to the paradigm polypeptide (i.e., a polypeptide that has a biological or pharmacological activity) such as naturally-occurring receptor-binding polypeptides, but have one or more peptide linkages optionally replaced by linkages such as —CH₂NH—, —CH₂S—, —CH₂—CH₂—, —CH═CH—(cis and trans), —CH₂SO—, —CH(OH)CH₂—, —COCH₂— etc., by methods well known in the art (Spatola, Peptide Backbone Modifications, Vega Data, 1(3):267 (1983); Spatola et al. Life Sci. 38:1243-1249 (1986); Hudson et al. Int. J. Pept. Res. 14:177-185 (1979); and Weinstein. B., 1983, Chemistry and Biochemistry, of Amino Acids, Peptides and Proteins, Weinstein eds, Marcel Dekker, New-York,). Such peptide mimetics may have significant advantages over naturally-occurring polypeptides including more economical production, greater chemical stability, enhanced pharmacological properties (e.g., half-life, absorption, potency, efficiency, etc.), reduced antigenicity and others.

While peptides may be effective in eliciting a biological activity in vitro, their effectiveness in vivo might be reduced by the presence of proteases. Serum proteases have specific substrate requirements. The substrate must have both L-amino acids and peptide bonds for cleavage. Furthermore, exopeptidases, which represent the most prominent component of theprotease activity in serum, usually act on the first peptide bond of the peptide and require a free N-terminus (Powell et al., Pharm. Res. 10:1268-1273 (1993)). In light of this, it is often advantageous to use modified versions of peptides. The modified peptides retain the structural characteristics of the original L-amino acid peptides that confer the desired biological activity of Ang(1-7) but are advantageously not readily susceptible to cleavage by protease and/or exopeptidases.

Systematic substitution of one or more amino acids of a consensus sequence with D-amino acid of the same type (e.g., D-lysine in place of L-lysine) may be used to generate more stable peptides. Thus, a peptide derivative or peptidomimetic of the present invention may be all L, all D or mixed D, L peptide, in either forward or reverse order. The presence of an N-terminal or C-terminal D-amino acid increases the in vivo stability of a peptide since peptidases cannot utilize a D-amino acid as a substrate (Powell et al., Pharm. Res. 10:1268-1273 (1993)). Reverse-D peptides are peptides containing D-amino acids, arranged in a reverse sequence relative to a peptide containing L-amino acids. Thus, the C-terminal residue of an L-amino acid peptide becomes N-terminal for the D-amino acid peptide, and so forth. Reverse D-peptides retain the same secondary conformation and therefore similar activity, as the L-amino acid peptides, but are more resistant to enzymatic degradation in vitro and in vivo, and thus can have greater therapeutic efficacy than the original peptide (Brady and Dodson, Nature 368:692-693 (1994); Jameson et al., Nature 368:744-746 (1994)). Similarly, a reverse-L peptide may be generated using standard methods where the C-terminus of the parent peptide becomes takes the place of the N-terminus of the reverse-L peptide. It is contemplated that reverse L-peptides of L-amino acid peptides that do not have significant secondary structure (e.g., short peptides) retain the same spacing and conformation of the side chains of the L-amino acid peptide and therefore often have the similar activity as the original L-amino acid peptide. Moreover, a reverse peptide may contain a combination of L- and D-amino acids. The spacing between amino acids and the conformation of the side chains may be retained resulting in similar activity as the original L-amino acid peptide.

In addition, constrained peptides comprising a consensus sequence or a substantially identical consensus sequence variation may be generated by methods well known in the art (Rizo and Gierasch, Ann. Rev. Biochem. 61:387-418 (1992)). For example, constrained peptides may be generated by adding cysteine residues capable of forming disulfide bridges and, thereby, resulting in a cyclic peptide. Cyclic peptides can be constructed to have no free N- or C-termini. Accordingly, they are not susceptible to proteolysis by exopeptidases, although they may be susceptible to endopeptidases, which do not cleave at peptide termini. The amino acid sequences of the peptides with N-terminal or C-terminal D-amino acids and of the cyclic peptides are usually identical to the sequences of the peptides to which they correspond, except for the presence of N-terminal or C-terminal D-amino acid residue, or their circular structure, respectively.

The invention also includes cyclized peptides. As used herein, a cyclic peptide has an intramolecular covalent bond between two non-adjacent residues. The intramolecular bond may be a backbone to backbone, side-chain to backbone or side-chain to side-chain bond (i.e., terminal functional groups of a linear peptide and/or side-chain functional groups of a terminal or interior residue may be linked to achieve cyclization). Typical intramolecular bonds include disulfide, amide and thioether bonds. A variety of means for cyclizing polypeptides are well known in the art, as are many other modifications that can be made to such peptides. For a general discussion, see International Patent Publication Nos. WO 01/53331 and WO 98/02452, the contents of which are incorporated herein by reference. Such cyclic bonds and other modifications can also be applied to the cyclic peptides and derivative compounds of this invention.

Cyclic peptides as described herein may comprise residues of L-amino acids, D-amino acids, or any combination thereof. Amino acids may be from natural or non-natural sources, provided that at least one amino group and at least one carboxyl group are present in the molecule; α- and β-amino acids are generally preferred. Cyclic peptides may also contain one or more rare amino acids (such as 4-hydroxyproline or hydroxylysine), organic acids or amides and/or derivatives of common amino acids, such as amino acids having the C-terminal carboxylate esterified (e.g., benzyl, methyl or ethyl ester) or amidated and/or having modifications of the N-terminal amino group (e.g., acetylation or alkoxycarbonylation), with or without any of a wide variety of side-chain modifications and/or substitutions (e.g., methylation, benzylation, t-butylation, tosylation, alkoxycarbonylation, and the like). Suitable derivatives include amino acids having an N-acetyl group (such that the amino group that represents the N-terminus of the linear peptide prior to cyclization is acetylated) and/or a C-terminal amide group (i.e., the carboxy terminus of the linear peptide prior to cyclization is amidated). Residues other than common amino acids that may be present with a cyclic peptide include, but are not limited to, penicillamine, β,β-tetramethylene cysteine, β,β-pentamethylene cysteine, β-mercaptopropionic acid, β,β-pentamethylene-β-mercaptopropionic acid, 2-mercaptobenzene, 2-mercaptoaniline, 2-mercaptoproline, ornithine, diaminobutyric acid, α-aminoadipic acid, m-aminomethylbenzoic acid and α,β-diaminopropionic acid.

Following synthesis of a linear peptide, with or without N-acetylation and/or C-amidation, cyclization may be achieved by any of a variety of techniques well known in the art. Within one embodiment, a bond may be generated between reactive amino acid side chains. For example, a disulfide bridge may be formed from a linear peptide comprising two thiol-containing residues by oxidizing the peptide using any of a variety of methods. Within one such method, air oxidation of thiols can generate disulfide linkages over a period of several days using either basic or neutral aqueous media. The peptide is used in high dilution to minimize aggregation and intermolecular side reactions. Alternatively, strong oxidizing agents such as I₂ and K₃Fe(CN)₆ can be used to form disulfide linkages. Those of ordinary skill in the art will recognize that care must be taken not to oxidize the sensitive side chains of Met, Tyr, Trp or His. Within further embodiments, cyclization may be achieved by amide bond formation. For example, a peptide bond may be formed between terminal functional groups (i.e., the amino and carboxy termini of a linear peptide prior to cyclization). Within another such embodiment, the linear peptide comprises a D-amino acid. Alternatively, cyclization may be accomplished by linking one terminus and a residue side chain or using two side chains, with or without an N-terminal acetyl group and/or a C-terminal amide. Residues capable of forming a lactam bond include lysine, ornithine (Orn), α-amino adipic acid, m-aminomethylbenzoic acid, α,β-diaminopropionic acid, glutamate or aspartate. Methods for forming amide bonds are generally well known in the art. Within one such method, carbodiimide-mediated lactam formation can be accomplished by reaction of the carboxylic acid with DCC, DIC, ED AC or DCCI, resulting in the formation of an O-acylurea that can be reacted immediately with the free amino group to complete the cyclization. Alternatively, cyclization can be performed using the azide method, in which a reactive azide intermediate is generated from an alkyl ester via a hydrazide. Alternatively, cyclization can be accomplished using activated esters. The presence of electron withdrawing substituents on the alkoxy carbon of esters increases their susceptibility to aminolysis. The high reactivity of esters of p-nitrophenol, N-hydroxy compounds and polyhalogenated phenols has made these “active esters” useful in the synthesis of amide bonds. Within a further embodiment, a thioether linkage may be formed between the side chain of a thiol-containing residue and an appropriately derivatized α-amino acid. By way of example, a lysine side chain can be coupled to bromoacetic acid through the carbodiimide coupling method (DCC, EDAC) and then reacted with the side chain of any of the thiol containing residues mentioned above to form a thioether linkage. In order to form dithioethers, any two thiol containing side-chains can be reacted with dibromoethane and diisopropylamine in DMF.

Substitution of non-naturally-occurring amino acids for natural amino acids in a subsequence of the peptides can also confer resistance to proteolysis. Such a substitution can, for instance, confer resistance to proteolysis by exopeptidases acting on the N-terminus without affecting biological activity. Examples of non-naturally-occurring amino acids include α,α-disubstituted amino acids, N-alkyl amino acids, C-α-methyl amino acids, β-amino acids, and β-methyl amino acids. Amino acids analogs useful in the present invention may include, but are not limited to, β-alanine, norvaline, norleucine, 4-aminobutyric acid, orithine, hydroxyproline, sarcosine, citrulline, cysteic acid, cyclohexylalanine, 2-aminoisobutyric acid, 6-aminohexanoic acid, t-butylglycine, phenylglycine, o-phosphoserine, N-acetyl serine, N-formylmethionine, 3-methylhistidine and other unconventional amino acids. Furthermore, the synthesis of peptides with non-naturally-occurring amino acids is routine in the art.

Another effective approach to confer resistance to peptidases acting on the N-terminal or C-terminal residues of a peptide is to add chemical groups at the peptide termini, such that the modified peptide is no longer a substrate for the peptidase. One such chemical modification is glycosylation of the peptides at either or both termini. Certain chemical modifications, in particular N-terminal glycosylation, have been shown to increase the stability of peptides in human serum (Powell et al., Pharm. Res. 10:1268-1273 (1993)). Other chemical modifications which enhance serum stability include, but are not limited to, the addition of an N-terminal alkyl group, consisting of a lower alkyl of from one to twenty carbons, such as an acetyl group, and/or the addition of a C-terminal amide or substituted amide group. In particular, the present invention includes modified peptides consisting of peptides bearing an N-terminal acetyl group and/or a C-terminal amide group.

Ang(1-7) polypeptides also include other types of peptide derivatives containing additional chemical moieties not normally part of the peptide, provided that the derivative retains the desired functional activity of the peptide. Examples of such derivatives include (1) N-acyl derivatives of the amino terminal or of another free amino group, wherein the acyl group may be an alkanoyl group (e.g., acetyl, hexanoyl, octanoyl) an aroyl group (e.g., benzoyl) or a blocking group such as F-moc (fluorenylmethyl-O—CO—); (2) esters of the carboxy terminal or of another free carboxy or hydroxyl group; (3) amide of the carboxy-terminal or of another free carboxyl group produced by reaction with ammonia or with a suitable amine; (4) phosphorylated derivatives; (5) derivatives conjugated to an antibody or other biological ligand and other types of derivatives; and (6) derivatives conjugated to a polyethylene glycol (PEG) chain.

Ang(1-7) polypeptides or analogs or derivatives of Ang(1-7) polypeptides may be obtained by any method of peptide synthesis known to those skilled in the art, including synthetic (e.g., exclusive solid phase synthesis, partial solid phase synthesis, fragment condensation, classical solution synthesis, native-chemical ligation) and recombinant techniques. For example, the peptides or peptides derivatives can be obtained by solid phase peptide synthesis, which in brief, consist of coupling the carboxyl group of the C-terminal amino acid to a resin (e.g., benzhydrylamine resin, chloromethylated resin, hydroxymethyl resin) and successively adding N-alpha protected amino acids. The protecting groups may be any such groups known in the art. Before each new amino acid is added to the growing chain, the protecting group of the previous amino acid added to the chain is removed. Such solid phase synthesis has been disclosed, for example, by Merrifield, J. Am. Chem. Soc. 85: 2149 (1964); Vale et al., Science 213:1394-1397 (1981), in U.S. Pat. Nos. 4,305,872 and 4,316,891, Bodonsky et al. Chem. Ind. (London), 38:1597 (1966); and Pietta and Marshall, Chem. Comm. 650 (1970) by techniques reviewed in Lubell et al. “Peptides” Science of Synthesis 21.11, Chemistry of Amides. Thieme, Stuttgart, 713-809 (2005). The coupling of amino acids to appropriate resins is also well known in the art and has been disclosed in U.S. Pat. No. 4,244,946. (Reviewed in Houver-Weyl, Methods of Organic Chemistry. Vol E22a. Synthesis of Peptides and Peptidomimetics, Murray Goodman, Editor-in-Chief, Thieme. Stuttgart. New York 2002).

Unless defined otherwise, the scientific and technological terms and nomenclature used herein have the same meaning as commonly understood by a person of ordinary skill to which this invention pertains. Generally, the procedures of cell cultures, infection, molecular biology methods and the like are common methods used in the art. Such standard techniques can be found in reference manuals such as, for example, Ausubel et al., Current Protocols in Molecular Biology, Wiley Interscience, New York, 2001; and Sambrook et al., Molecular Cloning: A Laboratory Manual, 3^{rd} edition, Cold Spring Harbor Laboratory Press, N.Y., 2001.

During any process of the preparation of an Ang(1-7) polypeptide or analog or derivative, it may be desirable to protect sensitive reactive groups on any of the molecule concerned. This may be achieved by means of conventional protecting groups such as those described in Protective Groups In Organic Synthesis by T. W. Greene & P. G. M. Wuts, 1991, John Wiley and Sons, New-York; and Peptides: chemistry and Biology by Sewald and Jakubke, 2002, Wiley-VCH, Wheinheim p. 142. For example, alpha amino protecting groups include acyl type protecting groups (e.g., trifluoroacetyl, formyl, acetyl), aliphatic urethane protecting groups (e.g., t-butyloxycarbonyl (BOC), cyclohexyloxycarbonyl), aromatic urethane type protecting groups (e.g., fluorenyl-9-methoxy-carbonyl (Fmoc), benzyloxycarbonyl (Cbz), Cbz derivatives) and alkyl type protecting groups (e.g., triphenyl methyl, benzyl). The amino acids side chain protecting groups include benzyl (for Thr and Ser), Cbz (Tyr, Thr, Ser, Arg, Lys), methyl ethyl, cyclohexyl (Asp, His), Boc (Arg, His, Cys) etc. The protecting groups may be removed at a convenient subsequent stage using methods known in the art.

Further, Ang(1-7) polypeptides, or Ang (1-7) polypeptide analogs or derivatives may be synthesized according to the FMOC protocol in an organic phase with protective groups. Desirably, the peptides are purified with a yield of 70% with high-performance liquid chromatography (HPLC) on a C18 chromatography column and eluted with an acetonitrile gradient of 10-60%. The molecular weight of a peptide can be verified by mass spectrometry (reviewed in Fields, G. B. “Solid-Phase Peptide Synthesis” Methods in Enzymology. Vol. 289, Academic Press, 1997).

Alternatively, Ang(1-7) polypeptides, or Ang(1-7) polypeptide analogs or derivatives may be prepared in recombinant systems using, for example, polynucleotide sequences encoding the polypeptides. It is understood that a polypeptide may contain more than one of the above-described modifications within the same polypeptide.

The Ang(1-7) polypeptides, analogs or derivatives described herein may also be formulated in pharmaceutical compositions to treat subjects with fibrotic disorders or susceptible to fibrosis.

Exemplary Angiotensin(1-7), Analogs and/or Derivatives

### Linear Angiotensin(1-7) Peptide and Peptide Analogs and Derivatives

As used herein, the term “angiotensin (1-7) peptide” refers to both naturally-occurring Angiotensin (1-7) and any functional equivalent, analog or derivative of naturally-occurring Angiotensin (1-7). As used herein, the terms “peptide” and “polypeptide” include both linear and cyclic peptide. The terms “angiotensin-(1-7)”, “Angiotensin-(1-7)”, and “Ang-(1-7)” are used interchangeably.

Naturally-Occurring Angiotensin (1-7)

Naturally-occurring Angiotensin (1-7) (also referred to as Ang-(1-7)) is a seven amino acid peptide shown below:

(SEQ ID NO: 1)
Asp¹-Arg²-Val³-Tyr⁴-Ile⁵-His⁶-Pro⁷

It is part of the renin-angiotensin system and is converted from a precursor, also known as Angiotensinogen, which is an α-2-globulin that is produced constitutively and released into the circulation mainly by the liver. Angiotensinogen is a member of the serpin family and also known as renin substrate. Human angiotensinogen is 452 amino acids long, but other species have angiotensinogen of varying sizes. Typically, the first 12 amino acids are the most important for angiotensin activity:

(SEQ ID NO: 4)
Asp¹-Arg²-Val³-Tyr⁴-Ile⁵-His⁶-Pro⁷-Phe⁸-
His⁹-Leu¹⁰-Val¹¹-Ile¹²

Different types of angiotensin may be formed by the action of various enzymes. For example, Angiotensin (1-7) is generated by action of Angiotensin-converting enzyme 2 (ACE 2).

Ang-(1-7) is an endogenous ligand for Mas receptors. Mas receptors are G-protein coupled receptor containing seven transmembrane spanning regions. As used herein, the term “angiotensin-(1-7) receptor’ encompasses the G Protein-Coupled Mas Receptors.

As used herein, the term “naturally-occurring Angiotensin (1-7)” includes any Angiotensin (1-7) peptide purified from natural sources and any recombinantly produced or chemically synthesized peptides that have an amino acid sequence identical to that of the naturally-occurring Angiotensin (1-7).

In various embodiments, the Angiotensin (1-7) peptide administered to a subject may be Asp-Arg-Val-Tyr-Ile, Asp-Arg-Val-Tyr-Ile-His, or Asp-Arg-Val-Tyr-Ile-His-Pro. The A(1-7) may be linear or cyclized in any suitable manner, such as those described in WO2008/018792, including but not limited to A(1-7) comprising a thioether bridge between positions 4 and 7.

Functional Equivalents, Analogs or Derivatives of Ang-(1-7)

In some embodiments, an angiotensin (1-7) peptide suitable for the present invention is a functional equivalent of naturally-occurring Ang-(1-7). As used herein, a functional equivalent of naturally-occurring Ang-(1-7) refers to any peptide that shares amino acid sequence identity to the naturally-occurring Ang-(1-7) and retain substantially the same or similar activity as the naturally-occurring Ang-(1-7). For example, in some embodiments, a functional equivalent of naturally-occurring Ang-(1-7) described herein has pro-angiogenic activity as determined using methods described herein or known in the art, or an activity such as nitric oxide release, vasodilation, improved endothelial function, antidiuresis, or one of the other properties discussed herein, that positively impacts angiogenesis. In some embodiments, a functional equivalent of naturally-occurring Ang-(1-7) described herein can bind to or activate an angiotensin-(1-7) receptor (e.g., the G protein-coupled Mas receptor) as determined using various assays described herein or known in the art. In some embodiments, a functional equivalent of Ang-(1-7) is also referred to as an angiotensin (1-7) analogue or derivative, or functional derivative.

Typically, a functional equivalent of angiotensin (1-7) shares amino acid sequence similarity to the naturally-occurring Ang-(1-7). In some embodiments, a functional equivalent of Ang-(1-7) according to the invention contains a sequence that includes at least 3 (e.g., at least 4, at least 5, at least 6, at least 7) amino acids from the seven amino acids that appear in the naturally-occurring Ang-(1-7), wherein the at least 3 (e.g., at least 4, at least 5, at least 6, or at least 7) amino acids maintain their relative positions and/or spacing as they appear in the naturally-occurring Ang-(1-7).

In some embodiments, a functional equivalent of Ang-(1-7) also encompass any peptide that contain a sequence at least about 50% (e.g., at least about 55%, 60%, 65%, 70%, 75%, 80%, 85%, 90%, 95%, 98% or more) identical to the amino acid sequence of naturally-occurring Ang-(1-7). Percentage of amino acid sequence identity can be determined by alignment of amino acid sequences. Alignment of amino acid sequences can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. Preferably, the WU-BLAST-2 software is used to determine amino acid sequence identity (Altschul et al., Methods in Enzymology 266, 460-480 (1996); http://blast.wustl/edu/blast/README.html). WU-BLAST-2 uses several search parameters, most of which are set to the default values. The adjustable parameters are set with the following values: overlap span=1, overlap fraction=0.125, word threshold (T)=11. HSP score (S) and HSP S2 parameters are dynamic values and are established by the program itself, depending upon the composition of the particular sequence, however, the minimum values may be adjusted and are set as indicated above.

In some embodiments, a functional equivalent, analogue or derivative of Ang-(1-7) is a fragment of the naturally-occurring Ang-(1-7). In some embodiments, a functional equivalent, analogue or derivative of Ang-(1-7) contains amino acid substitutions, deletions and/or insertions in the naturally-occurring Ang-(1-7). Ang-(1-7) functional equivalents, analogues or derivatives can be made by altering the amino acid sequences by substitutions, additions, and/or deletions. For example, one or more amino acid residues within the sequence of the naturally-occurring Ang-(1-7) (SEQ ID NO:1) can be substituted by another amino acid of a similar polarity, which acts as a functional equivalent, resulting in a silent alteration. Substitution for an amino acid within the sequence may be selected from other members of the class to which the amino acid belongs. For example, the positively charged (basic) amino acids include arginine, lysine, and histidine. The nonpolar (hydrophobic) amino acids include leucine, isoleucine, alanine, phenylalanine, valine, proline, tryptophane, and methionine. The uncharged polar amino acids include serine, threonine, cysteine, tyrosine, asparagine, and glutamine. The negatively charged (acid) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions. For example, the amino acid sequence of a peptide inhibitor can be modified or substituted.

Examples of Ang-(1-7) functional equivalents, analogues and derivatives are described in the section entitled “Exemplary Angiotensin(1-7) Peptides” below.

An angiotensin-(1-7) peptide can be of any length. In some embodiments, an angiotensin-(1-7) peptide according to the present invention can contain, for example, from 4-25 amino acids (e.g., 4-20, 4-15, 4-14, 4-13, 4-12, 4-11, 4-10, 4-9, 4-8, 4-7 amino acids). In some embodiments, the linear peptide contains 4, 5, 6, 7, 8, 9, 10, 11, 12, 13, 14, 16, 17, 18, 19, 20, 21, 22, 23, 24, or 25 amino acids.

In some embodiments, an angiotensin-(1-7) peptide contains one or more modifications to increase protease resistance, serum stability and/or bioavailability. In some embodiments, suitable modifications are selected from pegylation, acetylation, glycosylation, biotinylation, substitution with D-amino acid and/or un-natural amino acid, and/or cyclization of the peptide.

Exemplary Angiotensin-(1-7) Peptides

In certain aspects, the invention provides linear angiotensin-(1-7) peptides. As discussed above, the structure of naturally-occurring Ang-(1-7) is as follows:

(SEQ ID NO: 1)
Asp¹-Arg²-Val³-Tyr⁴-Ile⁵-His⁶-Pro⁷

The peptides and peptide analogs of the invention can be generally represented by the following sequence:

(SEQ ID NO: 5)
Xaa¹-Xaa²-Xaa³-Xaa⁴-Xaa⁵-Xaa⁶-Xaa⁷,

or a pharmaceutically acceptable salt thereof.

Xaa¹ is any amino acid or a dicarboxylic acid. In certain embodiments, Xaa¹ is Asp, Glu, Asn, Acpc (1-aminocyclopentane carboxylic acid), Ala, Me₂Gly (N,N-dimethylglycine), Pro, Bet (betaine, 1-carboxy-N,N,N-trimethylmethanaminium hydroxide), Glu, Gly, Asp, Sar (sarcosine) or Suc (succinic acid). In certain such embodiments, Xaa¹ is a negatively-charged amino acid, such as Asp or Glu, typically Asp.

Xaa² is Arg, Lys, Ala, Cit (citrulline), Orn (ornithine), acetylated Ser, Sar, D-Arg and D-Lys. In certain embodiments, Xaa² is a positively-charged amino acid such as Arg or Lys, typically Arg.

Xaa³ is Val, Ala, Leu, Nle (norleucine), Ile, Gly, Lys, Pro, HydroxyPro (hydroxyproline), Aib (2-aminoisobutyric acid), Acpc or Tyr. In certain embodiments, Xaa³ is an aliphatic amino acid such as Val, Leu, Ile or Nle, typically Val or Nle.

Xaa⁴ is Tyr, Tyr(PO₃), Thr, Ser, homoSer (homoserine), azaTyr (aza-α¹-homo-L-tyrosine) or Ala. In certain embodiments, Xaa⁴ is a hydroxyl-substituted amino acid such as Tyr, Ser or Thr, typically Tyr.

Xaa⁵ is Ile, Ala, Leu, norLeu, Val or Gly. In certain embodiments, Xaa⁵ is an aliphatic amino acid such as Val, Leu, Ile or Nle, typically Ile.

Xaa⁶ is His, Arg or 6-NH₂-Phe (6-aminophenylalaine). In certain embodiments, Xaa⁶ is a fully or partially positively-charged amino acid such as Arg or His.

Xaa⁷ is Cys, Pro or Ala.

In certain embodiments, one or more of Xaa¹-Xaa⁷ is identical to the corresponding amino acid in naturally-occurring Ang-(1-7). In certain such embodiments, all but one or two of Xaa¹-Xaa⁷ are identical to the corresponding amino acid in naturally-occurring Ang-(1-7). In other embodiments, all of Xaa¹-Xaa⁶ are identical to the corresponding amino acid in naturally-occurring Ang-(1-7).

In certain embodiments, Xaa³ is Nle. When Xaa³ is Nle, one or more of Xaa¹-Xaa² and Xaa⁴⁻⁷ are optionally identical to the corresponding amino acid in naturally-occurring Ang-(1-7). In certain such embodiments, all but one or two of Xaa¹-Xaa² and Xaa⁴⁻⁷ are identical to the corresponding amino acid in naturally-occurring Ang-(1-7). In other embodiments, all of Xaa¹-Xaa² and Xaa⁴⁻⁷ are identical to the corresponding amino acid in naturally-occurring Ang-(1-7), resulting in the amino acid sequence: Asp-Arg-Nle-Tyr-Ile-His-Pro (SEQ ID NO:2).

In certain embodiments, the peptide has the amino acid sequence Asp-Arg-Val-Ser-Ile-His-Cys (SEQ ID NO:3) or Asp-Arg-Val-ser-Ile-His-Cys (SEQ ID NO:6).

Exemplary Cyclic Angiotensin (1-7) Peptides

In certain aspects, the invention provides a cyclic angiotensin-(1-7) (Ang-(1-7)) peptide analog comprising a linkage, such as between the side chains of amino acids corresponding to positions Tyr⁴ and Pro⁷ in Ang. These peptide analogs typically comprise 7 amino acid residues, but can also include a cleavable sequence. As discussed in greater detail below, the invention includes fragments and analogs where one or more amino acids are substituted by another amino acid (including fragments). One example of such a fragment or analog is Asp¹-Arg²-Val³-Ser⁴-Ile⁵-His⁶-Cys⁷ (SEQ ID NO: 3), wherein a linkage is formed between Ser⁴ and Cys⁷.

Although the following section describes aspects of the invention in terms of a thioether bond linking residues at the 4- and 7-positions, it should be understood that other linkages (as described above) could replace the thioether bridge and that other residues could be cyclized. A thioether bridge is also referred to as a monosulfide bridge or, in the case of Ala-S-Ala, as a lanthionine bridge. Thioether bridge-containing peptides can be formed by two amino acids having one of the following formulas:

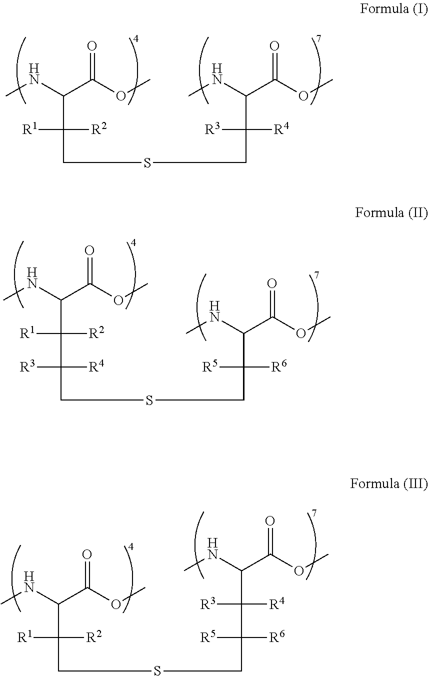

In these formulae, R¹, R², R³, R⁴, R⁵ and R⁶ are independently —H, an alkyl (e.g., C₁-C₆ alkyl, C₁-C₄ alkyl) or an aralkyl group, where the alkyl and aralkyl groups are optionally substituted with one or more halogen, —OH or —NRR′ groups (where R and R′ are independently —H or C₁-C₄ alkyl). In certain embodiments, R¹, R², R³, R⁴, R⁵ and R⁶ are each independently —H or —CH₃, such where all are —H.

In certain embodiments, the invention provides an Ang analog or derivative comprising a thioether bridge according to formula (I). Typically, R¹, R², R³ and R⁴ are independently selected from —H and —CH₃. Peptides comprising a thioether bridge according to formula (I) can be produced, for example, by lantibiotic enzymes or by sulfur extrusion of a disulfide. In one example, the disulfide from which the sulfur is extruded can be formed by D-cysteine in position 4 and L-cysteine in position 7 or by D-cysteine in position 4 and L-penicillamine in position 7 (see, e.g., Galande, Trent and Spatola (2003) Biopolymers 71, 534-551).

In other embodiments, the linkage of the two amino acids can be the bridges depicted in Formula (II) or Formula (III). Peptides comprising a thioether bridge according to Formula (II) can be made, for example, by sulfur extrusion of a disulfide formed by D-homocysteine in position 4 and L-cysteine in position 7. Similarly, peptides comprising a thioether bridge as in Formula (III) can be made, for example, by sulfur extrusion of a disulfide formed by D-cysteine in position 4 and L-homocysteine in position 7.

As discussed above, the Ang analogs and derivatives of the invention vary in length and amino acid composition. The Ang analogs and derivatives of the invention preferably have biological activity or are an inactive precursor molecule that can be proteolytically activated (such as how angiotensin(I), with 10 amino acids, is converted to active fragments by cleavage of 2 amino acids). The size of an Ang analog or derivative can vary but is typically between from about 5 to 10 amino acids, as long as the “core” pentameric segment comprising the 3-7 Nle-thioether-ring structure is encompassed. The amino acid sequence of an analog or derivative of the invention can vary, typically provided that it is biologically active or can become proteolytically activated. Biological activity of an analog or derivative can be determined using methods known in the art, including radioligand binding studies, in vitro cell activation assays and in vivo experiments. See, for example, Godeny and Sayeski, (2006) Am. J. Physiol. Cell. Physiol. 291:C1297-1307; Sarr et al., Cardiovasc. Res. (2006) 71:794-802; and Koziarz et al., (1933) Gen. Pharmacol. 24:705-713.

Representative cyclic Ang(1-7) analogs include a 4,7-cyclized analog designated [Cyc⁴⁻⁷]Ang(1-7), which is derived from natural Ang(1-7) (Asp¹-Arg²-Val³-Cyc⁴-Ile⁵-His⁶-Cyc⁷, SEQ ID NO: 7). These analogs can have one of the thioether bridges shown in Formulae (I)-(III) as the Cyc⁴⁻⁷ moiety, for example, where Cyc⁴ and Cyc⁷, taken together, are represented by Formula (I), such as where R′—R⁴ are each —H or —CH₃, typically —H.

The amino acids at positions other than 4 and 7 can be the same or different from the naturally-occurring peptide, typically provided that the analog retains a biological function. One example is Asp-Arg-Val-Ser-Ile-His-Cys (SEQ ID NO:3). For inactive precursors, biological function refers to one or both of an analog's susceptibility to angiotensin-converting enzymes that can cleave it to a biologically active fragment (e.g., Ang(1-7)) or the biological activity of the fragment itself. In certain embodiments, an Ang analog of the invention has no intrinsic function but inhibits the effects of one or more naturally-occurring angiotensin compounds.

Ang analogs and derivatives where only the length of the peptide is varied include the following:

a 4,7-cyclized analog designated [Cyc⁴⁻⁷]Ang-(1-7), which is derived from natural Ang-(1-7) (Asp¹-Arg²-Val³-Cyc⁴-Ile⁵-His⁶-Cyc⁷, SEQ ID NO:7).

a 4,7-cyclized analog designated [Nle³, Cyc⁴⁻⁷]Ang-(1-10), which is derived from natural Angiotensin I (Ang-(1-10)) (Asp¹-Arg²-Nle³-Cyc⁴-Ile⁵-His⁶-Cyc⁷-Phe⁸-His⁹-Leu¹⁰, SEQ ID NO:8);

a 4,7-cyclized analog designated [Nle³, Cyc⁴⁻⁷]Ang-(1-8), which is derived from natural Angiotensin II (Ang-(1-8)) (Asp¹-Arg²-Nle³-Cyc⁴-Ile⁵-His⁶-Cyc⁷-Phe⁸, SEQ ID NO:9);

a 4,7-cyclised analog designated [Nle³, Cyc⁴⁻⁷]Ang-(2-8), which is derived from natural Angiotensin III (Ang-(2-8)) (Arg²-Nle³-Cyc⁴-Ile⁵-His⁶-Cyc⁷-Phe⁸, SEQ ID NO:10);

a 4,7-cyclised analog designated [Nle³, Cyc⁴⁻⁷]Ang-(3-8), which is derived from natural Angiotensin IV (Ang-(3-8)) (Nle³-Cyc⁴-Ile⁵-His⁶-Cyc⁷-Phe⁸, SEQ ID NO:11);

a 4,7-cyclised analog designated [Nle³, Cyc⁴⁻⁷]Ang-(1-7) derived from natural Ang-(1-7) (Asp¹-Arg²-Nle³-Cyc⁴-Ile⁵-His⁶-Cyc⁷, SEQ ID NO:12); and
a 4,7-cyclised analog designated [Nle³, Cyc⁴⁻⁷]Ang-(1-9) derived from natural Ang-(1-9) (Asp¹-Arg²-Nle³-Cyc⁴-Ile⁵-His⁶-Cyc⁷-Phe⁸-His⁹, SEQ ID NO:13).
   These analogs can have one of the thioether bridges shown in Formulae (I)-(III) as the Cyc⁴⁻⁷ moiety, for example, where Cyc⁴ and Cyc⁷ are represented by Formula (I), such as where R¹-R⁴ are each —H or —CH₃, typically —H.

As compared to the amino acid sequence of the natural angiotensin peptide, the amino acids at positions 4 and 7 of the Cyc⁴⁻⁷ analog are modified to allow introduction of the thioether-ring structures shown above. In addition to the length of the Ang analogs, the amino acids at positions other than 3, 4 and 7 can be the same or different from the naturally-occurring peptide, typically provided that the analog retains a biological function. For analogs of inactive precursors, like [Cyc⁴⁻⁷]Ang-(1-10), biological function refers to one or both of an analog's susceptibility to angiotensin-converting enzymes that can cleave it to a biologically active fragment (e.g. Ang-(1-8) or Ang-(1-7)) or the biological activity of the fragment itself. In certain embodiments, an Ang analog or derivative of the invention has no intrinsic function but inhibits the effects of one or more naturally-occurring angiotensin compounds.

In certain embodiments, an Ang analog of the invention is represented by Formula (IV):

(IV, SEQ ID NO: 14)
Xaa¹-Xaa²-Xaa³-Cyc⁴-Xaa⁵-Xaa⁶-Cyc⁷

Xaa¹ is any amino acid, but typically a negatively-charged amino acid such as Glu or Asp, more typically Asp.

Xaa² is a positively-charged amino acid such as Arg or Lys, typically Arg.

Xaa³ is an aliphatic amino acid, such as Leu, Ile or Val, typically Val.

Cyc⁴ forms a thioether bridge in conjunction with Cyc⁷. Cyc⁴ can be a D-stereoisomer and/or a L-stereoisomer, typically a D-stereoisomer. Examples of Cyc⁴ (taken with Cyc⁷) are shown in Formulas (I), (II) and (III). Typically, the R groups in Formulae (I), (II) and (III) are —H or —CH₃, especially —H.

Xaa⁵ is an aliphatic amino acid, such as Leu, Ile or Val, typically Ile.

Xaa⁶ is His.

Cyc⁷ forms a thioether bridge in conjunction with Cyc⁴, such as in Formula (I), (II) or (III). Cyc⁷ can be a D-stereoisomer and/or a L-stereoisomer, typically a L-stereoisomer. Examples of Cyc⁷ (taken with Cyc⁴) are shown in Formulas (I), (II), (III) and (IV). Typically, the R groups in Formulas (I), (II), (III) and (IV) are —H or —CH₃, especially —H.

In certain embodiments, one or more of Xaa¹-Xaa⁶ (excluding Cyc⁴ and Cyc⁷) is identical to the corresponding amino acid in naturally-occurring Ang-(1-7). In certain such embodiments, all but one or two of Xaa¹-Xaa⁶ are identical to the corresponding amino acid in naturally-occurring Ang-(1-7). In other embodiments, all of Xaa¹-Xaa⁶ are identical to the corresponding amino acid in naturally-occurring Ang-(1-7).

In certain embodiments, Cyc⁴ and Cyc⁷ are independently selected from Abu (2-aminobutyric acid) and Ala (alanine), where Ala is present in at least one position. Thus, cyclic analogs can have a thioether linkage formed by -Ala⁴-S-Ala⁷- (Formula (I), where R¹-R⁴ are each —H); -Ala⁴-S-Abu⁷- (Formula (I): R¹-R³ are —H and R⁴ is —CH₃) or -Abu⁴-S-Ala⁷- (Formula (I): R¹, R³ and R⁴ are —H and R² is —CH₃). Specific examples of cyclic analogs comprise a -Abu⁴-S-Ala⁷- or -Ala⁴-S-Ala⁷- linkage.

In certain embodiments, the invention provides an Ang-(1-7) analog with a thioether-bridge between position 4 and position 7 having the amino acid sequence Asp-Arg-Val-Abu-Ile-His-Ala (SEQ ID NO:15) or the amino acid sequence Asp-Arg-Val-Ala-Ile-His-Ala (SEQ ID NO:16), which are represented by the following structural diagrams:

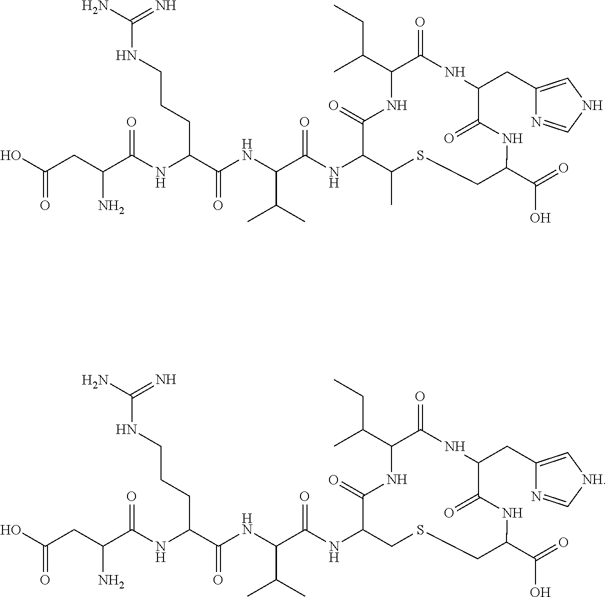 [SMILES: CCC(C)C1NC(=O)C(NC(=O)C(NC(=O)C(CCCNC(=N)N)NC(=O)C(N)CC(=O)O)C(C)C)C(C)SCC(C(=O)O)NC(=O)C(Cc2c[nH]cn2)NC1=O ; CCC(C)C1NC(=O)C(NC(=O)C(NC(=O)C(CCCNC(=N)N)NC(=O)C(N)CC(=O)O)C(C)C)CSCC(C(=O)O)NC(=O)C(Cc2c[nH]cn2)NC1=O]

In certain embodiments, an Ang analog or derivative of the invention is represented by Formula (V):

Xaa¹-Xaa²-Nle³-Cyc⁴-Xaa⁵-Xaa⁶-Cyc⁷-Xaa⁸-Xaa⁹-Xaa¹⁰ (V, SEQ ID NO:17)

As discussed above, one or more of Xaa¹, Xaa², Xaa⁸, Xaa⁹ and Xaa¹⁰ are absent in certain embodiments. For example, (1) Xaa¹⁰ is absent, (2) Xaa⁹ and Xaa¹⁰ are absent, (3) Xaa⁸, Xaa⁹ and Xaa¹⁰ are absent, (4) Xaa¹ is absent, (5) Xaa¹ and Xaa¹⁰ are absent, (6) Xaa¹, Xaa⁹ and Xaa¹⁰ are absent, (7) Xaa¹, Xaa⁸, Xaa⁹ and Xaa¹⁰ are absent, (8) Xaa¹ and Xaa² are absent, (9) Xaa¹, Xaa² and Xaa¹⁰ are absent, (10) Xaa¹, Xaa², Xaa⁹ and Xaa¹⁰ are absent, or (11) Xaa¹, Xaa², Xaa⁸, Xaa⁹ and Xaa¹⁰ are absent. For each of these embodiments, the remaining amino acids have the values described below.

Xaa¹, when present, is any amino acid, but typically a negatively charged amino acid such as Glu or Asp, more typically Asp.

Xaa², when present, is a positively charged amino acid such as Arg or Lys, typically Arg.

Nle³ is norleucine.

Cyc⁴ forms a thioether bridge in conjunction with Cyc⁷. Cyc⁴ can be a D-stereoisomer and/or a L-stereoisomer, typically a D-stereoisomer. Examples of Cyc⁴ (taken with Cyc⁷) are shown in Formulas (I), (II) and (III). Typically, the R groups in Formulae (I), (II) and (III) are —H or —CH₃, especially —H.

Xaa⁵ is an aliphatic amino acid, such as Leu, Nle, Ile or Val, typically Ile.

Xaa⁶ is His.

Cyc⁷ forms a thioether bridge in conjunction with Cyc⁴, such as in Formula (I), (II) or (III). Cyc⁷ can be a D-stereoisomer and/or a L-stereoisomer, typically a L-stereoisomer. Examples of Cyc⁷ (taken with Cyc⁴) are shown in Formulas (I), (II) and (III). Typically, the R groups in Formulae (I), (II) and (III) are —H or —CH₃, especially —H.

Xaa⁸, when present, is an amino acid other than Pro, typically Phe or Ile. In certain embodiments, Ile results in an inhibitor of Ang(1-8). In certain embodiments, Phe maintains the biological activity of Ang(1-8) or Ang(1-10).

Xaa⁹, when present, is His.

Xaa¹⁰, when present, is an aliphatic residue, for example, Ile, Val or Leu, typically Leu.

In certain embodiments, one or more of Xaa¹-Xaa¹⁰ (excluding Nle³, Cyc⁴ and Cyc⁷) is identical to the corresponding amino acid in naturally-occurring Ang (including Ang-(1-7), Ang(1-8), Ang(1-9), Ang(1-10), Ang(2-7), Ang(2-8), Ang(2-9), Ang(2-10), Ang(3-8), Ang(3-9) and Ang(3-10). In certain such embodiments, all but one or two of Xaa¹-Xaa¹⁰ (for those present) are identical to the corresponding amino acid in naturally-occurring Ang. In other embodiments, all of Xaa¹-Xaa¹⁰ (for those present) are identical to the corresponding amino acid in naturally-occurring Ang.

In certain embodiments, Cyc⁴ and Cyc⁷ are independently selected from Abu (2-aminobutyric acid) and Ala (alanine), where Ala is present at at least one position. Thus, encompassed are cyclic analogs comprising a thioether linkage formed by -Ala⁴-S-Ala⁷- (Formula (I), where R¹-R⁴ are each —H); -Ala⁴-S-Abu⁷- (Formula (I): R¹-R³ are —H and R⁴ is —CH₃) or -Abu⁴-S-Ala⁷- (Formula (I): R¹, R³ and R⁴ are —H and R² is —CH₃). Specific cyclic analogs comprise a -Abu⁴-S-Ala⁷- or -Ala⁴-S-Ala⁷- linkage.

In particular, the invention provides an Ang-(1-7) analog or derivative with a thioether-bridge between position 4 and position 7 having the amino acid sequence Asp-Arg-Nle-Abu-Ile-His-Ala (SEQ ID NO:18) or the amino acid sequence Asp-Arg-Nle-Ala-Ile-His-Ala (SEQ ID NO:19).

In another aspect, the invention provides an Ang-(1-8) analog or derivative with a thioether-bridge between position 4 and position 7 having Ang-(1-8) antagonistic activity, in particular an Ang(1-8) analog or derivative having the amino acid sequence Asp-Arg-Nle-Abu-Ile-His-Ala-Ile (SEQ ID NO:20), the amino acid sequence Asp-Arg-Nle-Ala-Ile-His-Ala-Ile (SEQ ID NO:21) or the amino acid sequence Asp-Arg-Nle-Abu-Ile-His-Ala-Ile (SEQ ID NO:22).

An alkyl group is a straight chained or branched non-aromatic hydrocarbon that is completely saturated. Typically, a straight chained or branched alkyl group has from 1 to about 20 carbon atoms, preferably from 1 to about 10. Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An aralkyl group is an alkyl group substituted by an aryl group. Aromatic (aryl) groups include carbocyclic aromatic groups such as phenyl, naphthyl, and anthracyl, and heteroaryl groups such as imidazolyl, thienyl, furyl, pyridyl, pyrimidyl, pyranyl, pyrazolyl, pyrrolyl, pyrazinyl, thiazolyl, oxazolyl, and tetrazolyl. Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl.

An alkenyl group is a straight chained or branched non-aromatic hydrocarbon that is includes one or more double bonds. Typically, a straight chained or branched alkenyl group has from 2 to about 20 carbon atoms, preferably from 2 to about 10. Examples of straight chained and branched alkenyl groups include ethenyl, n-propenyl, and n-butenyl.

Aromatic (aryl) groups include carbocyclic aromatic groups such as phenyl, naphthyl, and anthracyl, and heteroaryl groups such as imidazolyl, thienyl, furyl, pyridyl, pyrimidyl, pyranyl, pyrazolyl, pyrrolyl, pyrazinyl, thiazolyl, oxazolyl, and tetrazolyl. Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl.

Non-Peptide Analogs

The present invention also includes non-peptides analogs of Ang(1-7). Such analogs have can one or more functional properties of Ang(1-7), such as nitric oxide release, vasodilation, improved endothelial function, antidiuresis, or one of the other properties discussed herein.

An exemplary class of non-peptide analogs are angiotensin (1-7) receptor agonists. As used herein, the term “angiotensin-(1-7) receptor agonist” encompasses any molecule that has a positive impact in a function of an angiotensin-(1-7) receptor, in particular, the G-protein coupled Mas receptor. In some embodiments, an angiotensin-(1-7) receptor agonist directly or indirectly enhances, strengthens, activates and/or increases an angiotensin-(1-7) receptor (i.e., the Mas receptor) activity. In some embodiments, an angiotensin-(1-7) receptor agonist directly interacts with an angiotensin-(1-7) receptor (i.e., the Mas receptor). Such agonists can be peptidic or non-peptidic including, e.g., proteins, chemical compounds, small molecules, nucleic acids, antibodies, drugs, ligands, or other agents. In some embodiments, the angiotensin (1-7) receptor agonist is a non-peptidic agonist.

An exemplary class of angiotensin-(1-7) receptor agonists are 1-(p-thienylbenzyl)imidazoles. Examples of these non-peptide angiotensin-(1-7) receptor agonists are represented by Formula (VI):

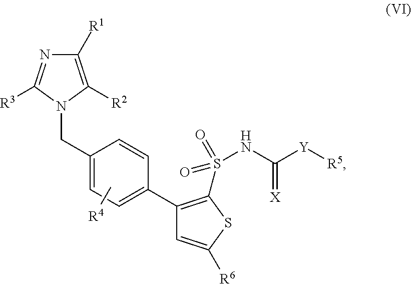

or pharmaceutically acceptable salts thereof, wherein:

R¹ is halogen, hydroxyl, (C₁-C₄)-alkoxy, (C₁-C₈)-alkoxy wherein 1 to 6 carbon atoms are replaced by the heteroatoms O, S, or NH (preferably by 0), (C₁-C₄)-alkoxy substituted by a saturated cyclic ether such as tetrahydropyran or tetrahydrofuran, O—(C₁-C₄)-alkenyl, O—(C₁-C₄)-alkylaryl, or aryloxy that is unsubstituted or substituted by a substituent selected from halogen, (C₁-C₃)-alkyl, (C₁-C₃)-alkoxy and trifluoromethyl;

R² is CHO, COOH, or (3) CO—O—(C₁-C₄)-alkyl;

R³ is (C₁-C₄)-alkyl or aryl;

R⁴ is hydrogen, halogen (chloro, bromo, fluoro), or (C₁-C₄)-alkyl;

X is oxygen or sulfur;

Y is oxygen or —NH—;

R⁵ is hydrogen, (C₁-C₆)-alkyl; or (C₁-C₄)-alkylaryl, where R⁵ is hydrogen when Y is —NH—; and

R⁶ is (C₁-C₅)-alkyl.

In certain embodiments, R¹ is not halogen when R² is COOH or CO—O—(C₁-C₄)-alkyl.

In some embodiments, an angiotensin-(1-7) receptor agonist is AVE 0991, 5-formyl-4-methoxy-2-phenyl-1 [[4-[2-(ethylaminocarbonylsulfonamido)-5-isobutyl-3-thienyl]-phenyl]-methyl]-imidazole, which is represented by the following structure:

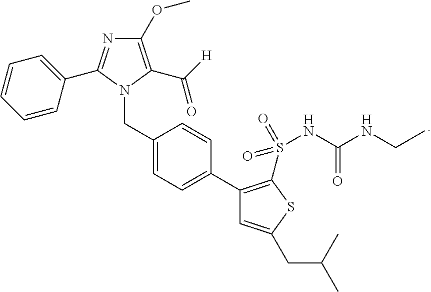 [SMILES: CCNC(=O)NS(=O)(=O)c1sc(CC(C)C)cc1-c1ccc(Cn2c(-c3ccccc3)nc(OC)c2C=O)cc1]

Another exemplary class of angiotensin-(1-7) receptor agonists are p-thienylbenzylamides. Examples of these non-peptide angiotensin-(1-7) receptor agonists are represented by Structural Formula (VII):

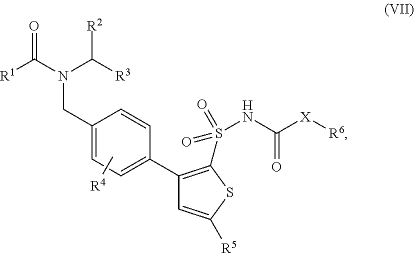

or a pharmaceutically acceptable salt thereof, wherein:

R¹ is (C₁-C₅)-alkyl that is unsubstituted or substituted by a radical chosen from NH₂, halogen, O—(C₁-C₃)-alkyl, CO—O—(C₁-C₃)-alkyl and CO₂H, (C₃-C₈)-cycloalkyl, (C₁-C₃)-alkyl-(C₃-C₈)-cycloalkyl, (C₆-C₁₀)-aryl that is unsubstituted or substituted by a radical chosen from halogen and O—(C₁-C₃)-alkyl, (C₁-C₃)-alkyl-(C₆-C₁₀)-aryl where the aryl radical is unsubstituted or substituted by a radical chosen from halogen and O—(C₁-C₃)-alkyl, (C₁-C₅)-heteroaryl, or (C₁-C₃)-alkyl-(C₁-C₅)-heteroaryl;

R² is hydrogen, (C₁-C₆)-alkyl that is unsubstituted or substituted by a radical chosen from halogen and O—(C₁-C₃)-alkyl, (C₃-C₈)-cycloalkyl, (C₁-C₃)-alkyl-(C₃-C₈)-cycloalkyl, (C₆-C₁₀)-aryl that is unsubstituted or substituted by a radical chosen from among halogen, O—(C₁-C₃)-alkyl and CO—O—(C₁-C₃)-alkyl, or (C₁-C₃)-alkyl-(C₆-C₁₀)-aryl that is unsubstituted or substituted by a radical chosen from halogen and O—(C₁-C₃)-alkyl;

R³ is hydrogen, COOH, or COO—(C₁-C₄)-alkyl;

R⁴ is hydrogen, halogen; or (C₁-C₄)-alkyl;

R⁵ is hydrogen or (C₁-C₆)-alkyl;

R⁶ is hydrogen, (C₁-C₆)-alkyl, (C₁-C₃)-alkyl-(C₃-C₈)-cycloalkyl, or (C₂-C₆)-alkenyl; and

X is oxygen or NH.

Additional examples of angiotensin-(1-7) receptor agonists are described in U.S. Pat. No. 6,235,766, the contents of which are incorporated by reference herein.

The Angiotensin (1-7) or analogs or derivatives thereof described above can be present as pharmaceutically acceptable salts. As used herein, “a pharmaceutically acceptable salt” refers to salts that retain the desired activity of the peptide or equivalent compound, but preferably do not detrimentally affect the activity of the peptide or other component of a system, which uses the peptide. Examples of such salts are acid addition salts formed with inorganic acids, for example, hydrochloric acid, hydrobromic acid, sulfuric acid, phosphoric acid, nitric acid, and the like. Salts may also be formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, pamoic acid, alginic acid, polyglutamic acid, and the like. Salts formed from a cationic material may utilize the conjugate base of these inorganic and organic acids. Salts may also be formed with polyvalent metal cations such as zinc, calcium, bismuth, barium, magnesium, aluminum, copper, cobalt, nickel and the like or with an organic cation formed from N,N′-dibenzylethylenediamine or ethylenediamine, or combinations thereof (e.g., a zinc tannate salt). The non-toxic, physiologically acceptable salts are preferred.

Therapeutically effective dosage amounts of angiotensin (1-7) peptides, including derivatives and analogs may be present in varying amounts in various embodiments. For example, in some embodiments, a therapeutically effective amount of an angiotensin (1-7) peptide may be an amount ranging from about 10-1000 mg (e.g., about 20 mg-1,000 mg, 30 mg-1,000 mg, 40 mg-1,000 mg, 50 mg-1,000 mg, 60 mg-1,000 mg, 70 mg-1,000 mg, 80 mg-1,000 mg, 90 mg-1,000 mg, about 10-900 mg, 10-800 mg, 10-700 mg, 10-600 mg, 10-500 mg, 100-1000 mg, 100-900 mg, 100-800 mg, 100-700 mg, 100-600 mg, 100-500 mg, 100-400 mg, 100-300 mg, 200-1000 mg, 200-900 mg, 200-800 mg, 200-700 mg, 200-600 mg, 200-500 mg, 200-400 mg, 300-1000 mg, 300-900 mg, 300-800 mg, 300-700 mg, 300-600 mg, 300-500 mg, 400 mg-1,000 mg, 500 mg-1,000 mg, 100 mg-900 mg, 200 mg-800 mg, 300 mg-700 mg, 400 mg-700 mg, and 500 mg-600 mg). In some embodiments, an angiotensin (1-7) peptide is present in an amount of or greater than about 10 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg. In some embodiments, an angiotensin (1-7) peptide is present in an amount of or less than about 1000 mg, 950 mg, 900 mg, 850 mg, 800 mg, 750 mg, 700 mg, 650 mg, 600 mg, 550 mg, 500 mg, 450 mg, 400 mg, 350 mg, 300 mg, 250 mg, 200 mg, 150 mg, or 100 mg.

In other embodiments, a therapeutically effective dosage amount may be, for example, about 0.001 mg/kg weight to 500 mg/kg weight, e.g., from about 0.001 mg/kg weight to 400 mg/kg weight, from about 0.001 mg/kg weight to 300 mg/kg weight, from about 0.001 mg/kg weight to 200 mg/kg weight, from about 0.001 mg/kg weight to 100 mg/kg weight, from about 0.001 mg/kg weight to 90 mg/kg weight, from about 0.001 mg/kg weight to 80 mg/kg weight, from about 0.001 mg/kg weight to 70 mg/kg weight, from about 0.001 mg/kg weight to 60 mg/kg weight, from about 0.001 mg/kg weight to 50 mg/kg weight, from about 0.001 mg/kg weight to 40 mg/kg weight, from about 0.001 mg/kg weight to 30 mg/kg weight, from about 0.001 mg/kg weight to 25 mg/kg weight, from about 0.001 mg/kg weight to 20 mg/kg weight, from about 0.001 mg/kg weight to 15 mg/kg weight, from about 0.001 mg/kg weight to 10 mg/kg weight.

In still other embodiments, a therapeutically effective dosage amount may be, for example, about 0.0001 mg/kg weight to 0.1 mg/kg weight, e.g. from about 0.0001 mg/kg weight to 0.09 mg/kg weight, from about 0.0001 mg/kg weight to 0.08 mg/kg weight, from about 0.0001 mg/kg weight to 0.07 mg/kg weight, from about 0.0001 mg/kg weight to 0.06 mg/kg weight, from about 0.0001 mg/kg weight to 0.05 mg/kg weight, from about 0.0001 mg/kg weight to about 0.04 mg/kg weight, from about 0.0001 mg/kg weight to 0.03 mg/kg weight, from about 0.0001 mg/kg weight to 0.02 mg/kg weight, from about 0.0001 mg/kg weight to 0.019 mg/kg weight, from about 0.0001 mg/kg weight to 0.018 mg/kg weight, from about 0.0001 mg/kg weight to 0.017 mg/kg weight, from about 0.0001 mg/kg weight to 0.016 mg/kg weight, from about 0.0001 mg/kg weight to 0.015 mg/kg weight, from about 0.0001 mg/kg weight to 0.014 mg/kg weight, from about 0.0001 mg/kg weight to 0.013 mg/kg weight, from about 0.0001 mg/kg weight to 0.012 mg/kg weight, from about 0.0001 mg/kg weight to 0.011 mg/kg weight, from about 0.0001 mg/kg weight to 0.01 mg/kg weight, from about 0.0001 mg/kg weight to 0.009 mg/kg weight, from about 0.0001 mg/kg weight to 0.008 mg/kg weight, from about 0.0001 mg/kg weight to 0.007 mg/kg weight, from about 0.0001 mg/kg weight to 0.006 mg/kg weight, from about 0.0001 mg/kg weight to 0.005 mg/kg weight, from about 0.0001 mg/kg weight to 0.004 mg/kg weight, from about 0.0001 mg/kg weight to 0.003 mg/kg weight, from about 0.0001 mg/kg weight to 0.002 mg/kg weight. In some embodiments, the therapeutically effective dose may be 0.0001 mg/kg weight, 0.0002 mg/kg weight, 0.0003 mg/kg weight, 0.0004 mg/kg weight, 0.0005 mg/kg weight, 0.0006 mg/kg weight, 0.0007 mg/kg weight, 0.0008 mg/kg weight, 0.0009 mg/kg weight, 0.001 mg/kg weight, 0.002 mg/kg weight, 0.003 mg/kg weight, 0.004 mg/kg weight, 0.005 mg/kg weight, 0.006 mg/kg weight, 0.007 mg/kg weight, 0.008 mg/kg weight, 0.009 mg/kg weight, 0.01 mg/kg weight, 0.02 mg/kg weight, 0.03 mg/kg weight, 0.04 mg/kg weight, 0.05 mg/kg weight, 0.06 mg/kg weight, 0.07 mg/kg weight, 0.08 mg/kg weight, 0.09 mg/kg weight, or 0.1 mg/kg weight. The effective dose for a particular individual can be varied (e.g., increased or decreased) over time, depending on the needs of the individual.

In some embodiments, a therapeutically effective dosage may be a dosage of 10 μg/kg/day, 50 μg/day μg/kg/day, 100 μg/kg/day, 250 μg/kg/day, 500 μg/kg/day, 1000 μg/kg/day or more. In various embodiments, the amount of Angiotensin (1-7) or an analog or derivative thereof or pharmaceutical salt thereof is sufficient to provide a dosage to a patient of between 0.01 μg/kg and 10 mg/kg; 0.1 μg/kg and 5 mg/kg; 0.1 μg/kg and 1000 μg/kg; 0.1 μg/kg and 900 μg/kg; 0.1 μg/kg and 900 μg/kg; 0.1 μg/kg and 800 μg/kg; 0.1 μg/kg and 700 μg/kg; 0.1 μg/kg and 600 μg/kg; 0.1 μg/kg and 500 μg/kg; or 0.1 μg/kg and 400 μg/kg.

Particular doses or amounts to be administered in accordance with the present invention may vary, for example, depending on the nature and/or extent of the desired outcome, on particulars of route and/or timing of administration, and/or on one or more characteristics (e.g., weight, age, personal history, genetic characteristic, lifestyle parameter, severity of cardiac defect and/or level of risk of cardiac defect, etc., or combinations thereof). Such doses or amounts can be determined by those of ordinary skill. In some embodiments, an appropriate dose or amount is determined in accordance with standard clinical techniques. For example, in some embodiments, an appropriate dose or amount is a dose or amount sufficient to reduce a disease severity index score by 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100% or more. For example, in some embodiments, an appropriate dose or amount is a dose or amount sufficient to reduce a disease severity index score by 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100%. Alternatively or additionally, in some embodiments, an appropriate dose or amount is determined through use of one or more in vitro or in vivo assays to help identify desirable or optimal dosage ranges or amounts to be administered.

### Dosing Schedules

Various embodiments may include differing dosing regimen. In some embodiments, the Angiotensin (1-7) or analog or derivative thereof is administered via continuous infusion. In some embodiments, the continuous infusion is intravenous. In other embodiments, the continuous infusion is subcutaneous. Alternatively or additionally, in some embodiments, the Angiotensin (1-7) or analog or derivative thereof is administered bimonthly, monthly, twice monthly, triweekly, biweekly, weekly, twice weekly, thrice weekly, daily, twice daily, or on another clinically desirable dosing schedule. The dosing regimen for a single subject need not be at a fixed interval, but can be varied over time, depending on the needs of the subject.

### Combination Therapies

In some embodiments, an Angiotensin (1-7) or analog or derivative thereof will be used as a part of a combination therapy. It is contemplated that any known therapeutic or treatment for one or more brain conditions may be used with one or more Angiotensin (1-7) or analogs or derivatives thereof, as disclosed herein. Exemplary compounds that may be used with one or more Angiotensin (1-7) or analogs or derivatives thereof as a combination therapy include, but are not limited to, corticosteroids, cyclophosphamide (Cytoxan), azathioprine (Imuran), N-acetylcysteine (NAC), KALYDECO™ (N-(2,4-di-tert-butyl-5-hydroxyphenyl)-1,4-dihydro-4-oxoquinoline-3-carboxamide), PULMOZYME® (Recombinant human deoxyribonuclease I), TOBI® (Tobramycin), and hypertonic saline.

### Delivery

Various delivery systems are known and can be used to administer peptides, peptide derivatives, peptidomimetics, non-peptide agonists or pharmaceutical compositions comprising polypeptides, peptide derivatives, peptidomimetics, and/or non-peptide agonists. The pharmaceutical compositions described herein can be administered by any suitable route including, intravenous or intramuscular injection, intraventricular or intrathecal injection (for central nervous system administration), orally, topically, subcutaneously, mucocutaneously, intrapulmonary (e.g., inhalation), subconjunctivally, intraocularly, or via intranasal, intradermal, sublingual, vaginal, rectal or epidural routes.

Other delivery systems well known in the art can be used for delivery of the pharmaceutical compositions described herein, for example via aqueous solutions, encapsulation in microparticules, or microcapsules. The pharmaceutical compositions of the present invention can also be delivered in a controlled release system. For example, a polymeric material can be used (see, e.g., Smolen and Ball, Controlled Drug Bioavailability, Drug product design and performance, 1984, John Wiley & Sons; Ranade and Hollinger, Drug Delivery Systems, pharmacology and toxicology series, 2003, 2^{nd} edition, CRRC Press). Alternatively, a pump may be used (Saudek et al., N. Engl. J. Med. 321:574 (1989)). The compositions described herein invention may also be coupled to a class of biodegradable polymers useful in achieving controlled release of the drug, for example, polylactic acid, polyorthoesters, cross-linked amphipathic block copolymers and hydrogels, polyhydroxy butyric acid, and polydihydropyrans.

In some embodiments, the Angiotensin (1-7) or analog or derivative thereof, or salt thereof is prepared as an aerosol formulation. Aerosol preparations are stable dispersions or suspensions of solid material and liquid droplets in a gaseous medium. The peptide delivered via this formulation is deposited in the airways by: gravitational sedimentation, inertial impaction, and diffusion. Exemplary aerosol device types that can be used to administer an aerosol formulation include jet or ultrasonic nebulizers and metered-dose inhalers (MDI), The metered-dose inhalers are most frequently used aerosol delivery system. In some embodiments, the pharmaceutical compositions comprise a therapeutically effective amount of an Angiotensin (1-7) peptide or analog or derivative thereof of at least 5 contiguous amino acids of A(1-7) in an aerosolized formulation.

In some embodiments, the Angiotensin (1-7) or analog or derivative thereof, or salt thereof is prepared as a powder, and can be administered via the pulmonary route by use of a dry-powder inhaler (DPI), which is designed to deliver drug/excipients powder to the lungs, or by insufflation using a syringe or similar device.

### Pharmaceutical Compositions

The pharmaceutical compositions can be in a variety of forms including oral dosage forms, topic creams, topical patches, iontophoresis forms, suppository, nasal spray and inhaler, eye drops, intraocular injection forms, depot forms, as well as injectable and infusible solutions. Methods for preparing pharmaceutical compositions are well known in the art.

Pharmaceutical compositions typically contain the active agent (e.g. peptide, peptide derivative, peptidomimetic, or non-peptide analog) in an amount effective to achieve the desired therapeutic effect while avoiding or minimizing adverse side effects. Pharmaceutically acceptable preparations and salts of the active agent are provided herein and are well known in the art. For the administration of polypeptides and the like, the amount administered desirably is chosen that is therapeutically effective with few to no adverse side effects. The amount of the therapeutic or pharmaceutical composition which is effective in the treatment of a particular disease, disorder or condition depends on the nature and severity of the disease, the target site of action, the subject's weight, special diets being followed by the subject, concurrent medications being used, the administration route and other factors that are recognized by those skilled in the art. The dosage can be adapted by the clinician in accordance with conventional factors such as the extent of the disease and different parameters from the subject. Typically, 0.0001 to 1,000 mg/kg/day is administered to the subject. Effective doses may be extrapolated from dose response curves derived from in vitro or animal model test systems.

In some embodiments, pharmaceutical compositions comprising Angiotensin (1-7) or analog or derivative thereof may be made up in a solid form (including granules, powders or suppositories), in aerosolized form, or in a liquid form (e.g., solutions, suspensions, or emulsions). The pharmaceutical compositions may be applied in a variety of solutions. Suitable solutions for use in accordance with the invention are sterile, dissolve sufficient amounts of the Angiotensin (1-7) or analog or derivative thereof, and are not harmful for the proposed application.

As described above, pharmaceutical compositions desirably include an Angiotensin (1-7) peptide, peptide derivative, peptidomimetic, and/or non-peptide agonist combined with a pharmaceutically acceptable carrier. The term carrier refers to diluents, adjuvants, excipients or vehicles with which the peptide, peptide derivative, peptidomimetic, and/or non-peptide agonist is administered. Such pharmaceutical carriers include sterile liquids such as water and oils including mineral oil, vegetable oil (e.g., soybean oil or corn oil), animal oil or oil of synthetic origin. Aqueous glycerol and dextrose solutions as well as saline solutions may also be employed as liquid carriers of the pharmaceutical compositions of the present invention. The choice of the carrier depends on factors well recognized in the art, such as the nature of the peptide, peptide derivative or peptidomimetic, its solubility and other physiological properties as well as the target site of delivery and application. Examples of suitable pharmaceutical carriers are described in Remington: The Science and Practice of Pharmacy by Alfonso R. Gennaro, 2003, 21^{th} edition, Mack Publishing Company. Moreover, suitable carriers for oral administration are known in the art and are described, for example, in U.S. Pat. Nos. 6,086,918, 6,673,574, 6,960,355, and 7,351,741 and in WO2007/131286, the disclosures of which are hereby incorporated by reference.

Further pharmaceutically suitable materials that may be incorporated in pharmaceutical preparations include absorption enhancers including those intended to increase paracellular absorption, excipients, pH regulators and buffers, osmolarity adjusters, preservatives, stabilizers, antioxidants, surfactants, thickeners, emollient, dispersing agents, flavoring agents, coloring agents, and wetting agents.

Examples of suitable pharmaceutical excipients include, water, glucose, sucrose, lactose, glycol, ethanol, glycerol monostearate, gelatin, starch flour (e.g., rice flour), chalk, sodium stearate, malt, sodium chloride, and the like. The pharmaceutical compositions comprising Ang(1-7) polypeptides can take the form of solutions, capsules, tablets, creams, gels, powders sustained release formulations and the like. The composition can be formulated as a suppository, with traditional binders and carriers such as triglycerides (see Remington: The Science and Practice of Pharmacy by Alfonso R. Gennaro, 2003, 21^{th} edition, Mack Publishing Company). Such compositions contain a therapeutically effective amount of the therapeutic composition, together with a suitable amount of carrier so as to provide the form for proper administration to the subject. The formulations are designed to suit the mode of administration and the target site of action (e.g., a particular organ or cell type).

Examples of fillers or binders that may be used in accordance with the present invention include acacia, alginic acid, calcium phosphate (dibasic), carboxymethylcellulose, carboxymethylcellulose sodium, hydroxyethylcellulose, hydroxypropylcellulose, hydroxypropylmethylcellulose, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth microcrystalline cellulose, starch, and zein. In certain embodiments, a filler or binder is microcrystalline cellulose.

Examples of disintegrating agents that may be used include alginic acid, carboxymethylcellulose, carboxymethylcellulose sodium, hydroxypropylcellulose (low substituted), microcrystalline cellulose, powdered cellulose, colloidal silicon dioxide, sodium croscarmellose, crospovidone, methylcellulose, polacrilin potassium, povidone, sodium alginate, sodium starch glycolate, starch, disodium disulfite, disodium edathamil, disodium edetate, disodiumethylenediaminetetraacetate (EDTA) crosslinked polyvinylpyrollidines, pregelatinized starch, carboxymethyl starch, sodium carboxymethyl starch, microcrystalline cellulose.

Examples of lubricants include calcium stearate, canola oil, glyceryl palmitostearate, hydrogenated vegetable oil (type I), magnesium oxide, magnesium stearate, mineral oil, poloxamer, polyethylene glycol, sodium lauryl sulfate, sodium stearate fumarate, stearic acid, talc and, zinc stearate, glyceryl behapate, magnesium lauryl sulfate, boric acid, sodium benzoate, sodium acetate, sodium benzoate/sodium acetate (in combination), DL-leucine.

Examples of silica flow conditioners include colloidal silicon dioxide, magnesium aluminum silicate and guar gum. Another most preferred silica flow conditioner consists of silicon dioxide.

Examples of stabilizing agents include acacia, albumin, polyvinyl alcohol, alginic acid, bentonite, dicalcium phosphate, carboxymethylcellulose, hydroxypropylcellulose, colloidal silicon dioxide, cyclodextrins, glyceryl monostearate, hydroxypropyl methylcellulose, magnesium trisilicate, magnesium aluminum silicate, propylene glycol, propylene glycol alginate, sodium alginate, carnauba wax, xanthan gum, starch, stearate(s), stearic acid, stearic monoglyceride and stearyl alcohol.

Pharmaceutical compositions comprising Angiotensin (1-7) or analogs or derivatives thereof also include compositions formulated as neutral or salt forms. Pharmaceutically acceptable salts include those that form with free amino groups and those that react with free carboxyl groups. Non-toxic alkali metal, alkaline earth metal, and ammonium salts commonly used in the pharmaceutical industry include sodium, potassium, lithium, calcium, magnesium, barium, ammonium, and protamine zinc salts, which are prepared by methods well known in the art. Also included are non-toxic acid addition salts, which are generally prepared by reacting the compounds of the present invention with suitable organic or inorganic acid. Representative salts include the hydrobromide, hydrochloride, valerate, oxalate, oleate, laureate, borate, benzoate, sulfate, bisulfate, acetate, phosphate, tysolate, citrate, maleate, fumarate, tartrate, succinate, napsylate salts, and the like.

In some embodiments, suitable acids which are capable of forming salts with Angiotensin (1-7) or analogs or derivatives thereof include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid and the like. Suitable bases capable of forming salts with A(1-7) include inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine and the like) and optionally substituted ethanol-amines (e.g., ethanolamine, diethanolamine and the like).

In some embodiments, the pharmaceutical compositions are combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compositions of this invention may be dissolved in saline, water, polyethylene glycol, propylene glycol, carboxymethyl cellulose colloidal solutions, hydroxyethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The pharmaceutical compositions described herein may contain modifications of Ang(1-7) peptides or analogs or derivatives such that they are more stable once administered to a subject (i.e., once administered it has a longer half-life or longer period of effectiveness as compared to the unmodified form). Such modifications are well known to those skilled in the art to which this invention pertains (e.g., polyethylene glycol derivatization a.k.a. PEGylation, microencapsulation, etc).

In certain embodiments, methods of treating fibrosis that occurs in pulmonary tissue (i.e. lung) are described. The methods comprise the step of administering a composition comprising an Angiotensin (1-7) polypeptide to a subject suffering from or susceptible to a fibrotic disorder of the lung.

In certain embodiments of the methods described herein, the disorder to be treated by administration of an Angiotensin (1-7) or analog or derivative thereof is pulmonary fibrosis, pulmonary hypertension, COPD, asthma, and/or cystic fibrosis. In certain embodiments, methods of reducing or preventing fibrosis are described. The methods comprise administering a composition comprising an Angiotensin (1-7) polypeptide or analog or derivative thereof to a subject susceptible to fibrosis. In certain embodiments, the subject is susceptible to fibrosis caused by post-surgical adhesion formation.

In certain embodiments of the methods described herein, the Angiotensin (1-7) polypeptide analog or derivative is linear. In certain embodiments of the methods described herein, the Angiotensin (1-7) polypeptide analog or derivative is cyclic. The synthesis and structure of particular cyclic angiotensin polypeptides are disclosed in U.S. Patent Publication No. 2010055146, incorporated herein by reference in its entirety.

### Kits

In some embodiments, the present invention further provides kits or other articles of manufacture which contains an Ang (1-7) peptide, an angiotensin (1-7) receptor agonist or a formulation containing the same and provides instructions for its reconstitution (if lyophilized) and/or use. Kits or other articles of manufacture may include a container, a syringe, vial and any other articles, devices or equipment useful in administration (e.g., subcutaneous, by inhalation). Suitable containers include, for example, bottles, vials, syringes (e.g., pre-filled syringes), ampules, cartridges, reservoirs, or lyo-jects. The container may be formed from a variety of materials such as glass or plastic. In some embodiments, a container is a pre-filled syringe. Suitable pre-filled syringes include, but are not limited to, borosilicate glass syringes with baked silicone coating, borosilicate glass syringes with sprayed silicone, or plastic resin syringes without silicone.

Typically, the container may holds formulations and a label on, or associated with, the container that may indicate directions for reconstitution and/or use. For example, the label may indicate that the formulation is reconstituted to concentrations as described above. The label may further indicate that the formulation is useful or intended for, for example, subcutaneous administration. In some embodiments, a container may contain a single dose of a stable formulation containing an Ang (1-7) peptide or angiotensin (1-7) receptor agonist. In various embodiments, a single dose of the stable formulation is present in a volume of less than about 15 ml, 10 ml, 5.0 ml, 4.0 ml, 3.5 ml, 3.0 ml, 2.5 ml, 2.0 ml, 1.5 ml, 1.0 ml, or 0.5 ml. Alternatively, a container holding the formulation may be a multi-use vial, which allows for repeat administrations (e.g., from 2-6 administrations) of the formulation. Kits or other articles of manufacture may further include a second container comprising a suitable diluent (e.g., BWFI, saline, buffered saline). Upon mixing of the diluent and the formulation, the final protein concentration in the reconstituted formulation will generally be at least 1 mg/ml (e.g., at least 5 mg/ml, at least 10 mg/ml, at least 20 mg/ml, at least 30 mg/ml, at least 40 mg/ml, at least 50 mg/ml, at least 75 mg/ml, at least 100 mg/ml). Kits or other articles of manufacture may further include other materials desirable from a commercial and user standpoint, including other buffers, diluents, filters, needles, syringes, and package inserts with instructions for use. In some embodiments, kits or other articles of manufacture may include an instruction for self-administration.

### EXAMPLES

### Example 1

### Treatment of Cystic Fibrosis

This example demonstrates that an angiotensin (1-7) peptide may be used to treat cystic fibrosis.

Experiments are performed using the mouse model of chronic lung infection of P. aeruginosa as disclosed by Hoffman et al. (2005) Infection and Immunity (73)4: 2540-2514; the materials and methods and results sections of which are incorporated herein by reference. The histopathology of lungs from the mouse model of chronic P. aeruginosa is comparable to the lungs of patients with cystic fibrosis and is an animal model for cystic fibrosis. Briefly, a stable infection of mucoid bacteria that express quorum sensing factors is introduced into the lung. The mucoid bacteria are cultured in ox broth supplemented with 1% glycerol. The cells are then harvested and colony forming units are counted and adjusted to appropriate challenge inoculum by dilution in a purified alginate solution.

Female and male homozygotic (CFTR^{-/-}) transgenic Cftr^{tm1Unc}-TgN(FABPCFTR) mice, available from Jackson Laboratories, are utilized to test the therapeutic effects of angiotensins for cystic fibrosis. The mice are approximately 12 to 20 weeks old when testing begins. The mice are anesthetized and tracheotomized, followed by intratracheal challenge with 40 μl of planktonic mucoid (e.g., NH57388A), nonmucoid (e.g. NH57388C)P. aeruginosa strains, resulting in about ca. 4×10⁶ to about c.a. 4×10⁷ CFU/lung. The challenge is performed with a bead curved needle.

The whole lung of each mouse is excised aseptically and homogenized in 5 ml of sterile 0.9% saline, and 100 μl of appropriately serial diluted lung homogenates samples are plated on blood agar plates (BAP), incubated at 37° C., and inspected for P. aeruginosa colonies (i.e. CFU) after 35 to 40 hours.

Randomly selected mice are used for lung histopathology. The lungs are fixed in formalin buffer for at least one week, followed by embedding in paraffin wax, then cut into 5 μm sections. Mounted sections are stained with hematoxin and eosin (HE) combined with Alcian blue-periodic acid-Schiff stain for exopolysaccharides. The cellular changes are assigned to acute or chronic inflammation groups by a scoring system based on the proportion of polymorphonuclear leukocytes (PMN) and mononuclear leukocytes (MN) in the inflammatory foci. Acute inflammation predominately affects PMN whereas chronic inflammation affects predominately MN.

To measure alginate content, mouse lung homogenate (500 μl) is extracted with ice-cold ethanol (2 ml) and resuspended in sterile 0.9% saline (500 μl). The content of uronic acid (alginate) is quantified by a carbazole-borate assay. Lung homogenate from mice challenged with 0.9% saline in purified alginate is used as a blank.

Compositions comprising Angiotensin (1-7) polypeptide and/or its analogs and derivatives are administered prior to, concomitantly, and/or after inoculation with P. aeruginosa to reduce the symptoms of cystic fibrosis.

### Example 2

### Treatment of Pulmonary Fibrosis

Bleomycin (BLEO) administration induces pulmonary fibrosis and is an animal model for pulmonary fibrosis in humans. Ang 1-7 affects BLEO induced alterations in lung mechanics, pulmonary hemodynamics, and right ventricular remodeling in rats.

Wistar rats are fed normal chow and housed under standard laboratory conditions. All animals are allowed to acclimate for at least 7 days prior to minipump implantation, BLEO instillation and study enrollment. Each animal is implanted a preloaded osmotic minipump (Alzet 2ML2) interfaced with a venous catheter containing vehicle (0.9% NaCl) or one of four doses (20.83, 69.44, 208.33, 625 ng/kg/min) of TXA127 dissolved in vehicle.

The catheter is inserted into the femoral vein, advanced to the thoracic vena cava and secured in place. After patency is verified, the minipump is secured in a subcutaneous location on the animal's back. The following day, each animal receives an intratracheal instillation of BLEO (1.25 mpk, i.t; 0.133 ml/100 g BW) dissolved in vehicle or its vehicle (0.9% NaCl). Animals subjected to daily weight and health assessments from the inception of dosing throughout the duration of study.

On the final day of the study, rats are anesthetized with 5% isoflurane in a closed chamber carried by 100% Oxygen. Rats are then transferred to a nosecone anesthesia system to breathe normobaric, normoxic (79% N₂, 21% O₂) gas for determination of arterial blood gases obtained via direct carotid arterial cannulation. Rats are then administered pancuronium bromide (1.5%, i.p.) to inhibit voluntary respiratory efforts and then transferred to a FlexiVent ventilator for the direct assessment of pulmonary mechanics (pressure volume relationships). Following the conclusion of pulmonary function measurements, the rats are placed on a positive pressure ventilator for the determination of steady-state pulmonary arterial hemodynamics by direct pulmonary arterial catheterization. Following the completion of hemodynamic evaluations, animals are instilled with 10 ml of sterile PBS, and BALF collected. Finally, terminal blood is acquired and placed on ice.

The heart and lungs are harvested, immediately immersed in ice-cold (4° C.) 0.9% NaCl and then subjected to morphological analyses. Biopsies of right ventricle, pulmonary arterial trunk, and lung tissue are obtained and immediately flash-frozen in liquid nitrogen and stored at −80° C. for later analysis. An entire lobe of lung and mid-transverse section of right ventricle are obtained and fixed. Blood will be appropriately processed for production of plasma and serum for endpoint Ang(1-7) and biomarker evaluation, respectively.

A separate sample of whole blood is evaluated for arterial hematocrit. Fixed lung sections are transferred to 70% EtOH for subsequent paraffin embedding, sectioning, and staining. Quantitative analysis for lung fibrosis using Masson's Trichrome staining and image analysis is performed.

Levels of expressed profibrotic markers, CTGF, Collagen lal, TGF-β₁, and TIMP-1, are analyzed in pulmonary tissue and levels of TNFα are measured in bronchoalveoolar lavage fluid. Lung fibrosis is measured by a conventional hydroxyproline assay, a surrogate marker of collagen deposition.

### Example 3

### Treatment of Non Alcoholic Steatohepatitis (NASH)

A single injection of streptozotocin (STZ) is known to induce a non-alcoholic steatohepatitis (NASH)-like condition in mice and serves as a model of the disease in human subjects.

In this example, NASH was induced in 48 male mice by a single subcutaneous injection of streptozotocin (STZ, Sigma-Aldrich, USA) solution 2 days after birth and feeding with high fat diet (HFD, 57 kcal % fat, cat#HFD32, CLEA Japan, Japan) after 4 weeks of age. The mice were randomized into 6 groups of 8 mice at 7 weeks of age according to Table 1 below. Eight male littermates, fed with normal diet without STZ treatment, were used for the Normal group.

TABLE 1
Experimental Design

| | No. | | Test | Dose | | Volume | | Sacrifice |
|---|---|---|---|---|---|---|---|---|
| Group | mice | Mice | substance | (/kg) | | (mL/kg) | Regimens | (wks of age) |
| 1 | 8 | Normal | — | — | | — | — | 10 |
| 2 | 8 | STAM | Vehicle | — | | 5 | — | 10 |
| 3 | 8 | STAM | TXA127 | 30 | μg | 5 | SQ, QD, 7 wks-10 wks | 10 |
| 4 | 8 | STAM | TXA127 | 100 | μg | 5 | SQ, QD, 7 wks-10 wks | 10 |
| 5 | 8 | STAM | TXA127 | 300 | μg | 5 | SQ, QD, 7 wks-10 wks | 10 |
| 6 | 8 | STAM | TXA127 | 1000 | μg | 5 | SQ, QD, 7 wks-10 wks | 10 |
| 7 | 8 | STAM | Telmisartan | 15 | mg | 10 | Oral, QD, 8 wks-10 wks | 10 |

Group Descriptions

Group 1 (normal) consisted of eight normal mice fed with a normal diet ad libitum without any treatment. Group 2 (vehicle) consisted of eight NASH mice that were subcutaneously administered vehicle at a volume of 5 mL/kg once daily from 7 to 10 weeks of age. Group 3 (TXA127-30 μg) consisted of eight NASH mice that were subcutaneously administered vehicle supplemented with TXA127 at a dose of 30 μg/5 mL/kg once daily from 7 to 10 weeks of age. Group 4 (TXA127-100 μg) consisted of eight NASH mice that were subcutaneously administered vehicle supplemented with TXA127 at a dose of 100 μg/5 mL/kg once daily from 7 to 10 weeks of age. Group 5 (TXA127-300 μg) consisted of eight NASH mice that were subcutaneously administered vehicle supplemented with TXA127 at a dose of 300 μg/5 mL/kg once daily from 7 to 10 weeks of age. Group 6 (TXA127-1,000 μg) consisted of eight NASH mice that were subcutaneously administered vehicle supplemented with TXA127 at a dose of 1,000 μg/5 mL/kg once daily from 7 to 10 weeks of age. Group 7 consisted of eight NASH mice that were orally administered pure water supplemented with Telmisartan at a dose of 15 mg/10 mL/kg once daily from 8 to 10 weeks of age.

Vehicle (saline) and TXA127 were administered via subcutaneous administration to the mice in a volume of 5 mL/kg body weight. Telmisartan was administered by oral route to the mice in a volume of 10 mL/kg body weight. TXA127 was dissolved in saline, and Telmisartan (MICARDIS®) was purchased from Boehringer Ingelheim GmbH and was dissolved in pure water. TXA127 was administered once daily at the doses of 30, 100, 300 or 1000 μg/kg body weight. Telmisartan was administered once daily at the dose of 15 mg/kg body weight.

C57BL/6 mice (15-day-pregnant female) were obtained from Charles River Laboratories Japan (Kanagawa, Japan). All animals used in this study were housed and cared for in accordance with the Japanese Pharmacological Society Guidelines for Animal Use. The animals were maintained in a SPF facility under controlled conditions of temperature (23±2° C.), humidity (45±10%), lighting (12-hour artificial light and dark cycle; light from 8:00 to 20:00) and air exchange. A high pressure (20±4 Pa) was maintained in the experimental room to prevent contamination within the facility. Sterilized solid HFD was provided ad libitum, being placed in the metal lid on top of the cage. Distilled water was provided ad libitum from a water bottle equipped with a rubber stopper and a sipper tube. Water bottles were replaced once a week, cleaned and sterilized in an autoclave and reused.

Blood Glucose Measurement

Non-fasting whole blood glucose levels were measured in whole blood samples using G Checker (Sanko Junyaku, Japan). For plasma biochemistry, blood was collected in polypropylene tubes with anticoagulant (Novo-Heparin, Mochida Pharmaceutical, Japan) and centrifuged at 1,000×g for 15 minutes at 4° C. The supernatant was collected and stored at −80° C. until use. The plasma levels of ALT, AST, and ALP were measured by FUJI DRI-CHEM 7000 (Fuji Film, Japan).

Liver Hydroxyproline Measurement

To quantify liver hydroxyproline content, frozen livers (40-60 mg) were minced and defatted in acetone for 30 minutes at room temperature. After centrifugation, the pellets were air-dried and dissolved in 400 μL of 2N NaOH at 65° C. The liver lysates were autoclaved at 121° C. for 20 minutes. The samples were then acid-hydrolyzed with 400 μL of 6N HCl at 121° C. for 20 minutes, and neutralized with 400 μL of 10 mg/mL activated carbon in 4N NaOH. The neutralized samples were buffered with 2.2 M acetic acid/0.48 M citric acid buffer and centrifuged to obtain the supernatant. In order to construct a standard curve of hydroxyproline, serial dilutions of trans-4-hydroxy-L-proline standard (Sigma, USA) were prepared starting at 16 μg/mL. Five hundred μL of the supernatant and standard were added to 500 μL chloramine T in 10% n-propanol/acetate-citrate buffer and incubated for 25 minutes at room temperature. Five hundred μL of Ehrlich's solution was added, mixed, and incubated at 65° C. for 20 minutes. After samples were cooled on ice and centrifuged to collect the supernatant, the optical density of each supernatant and standard was measured at 560 nm and the concentration of liver hydroxyproline was calculated form the hydroxyproline standard curve. Protein concentrations of each supernatant were determined using a BCA protein assay kit (Thermo Scientific, USA). Liver hydroxyproline content was normalized by total protein in the liver.

Sirius Red-Staining

For quantitative analysis of fibrosis areas, bright field images of Sirius red-stained sections were captured using a digital camera (DFC280, Leica, Germany) around central veins at 200-fold magnification, and the positive areas in 5 fields/section were quantified using ImageJ software (National Institute of Health, USA).

Blood Glucose Results

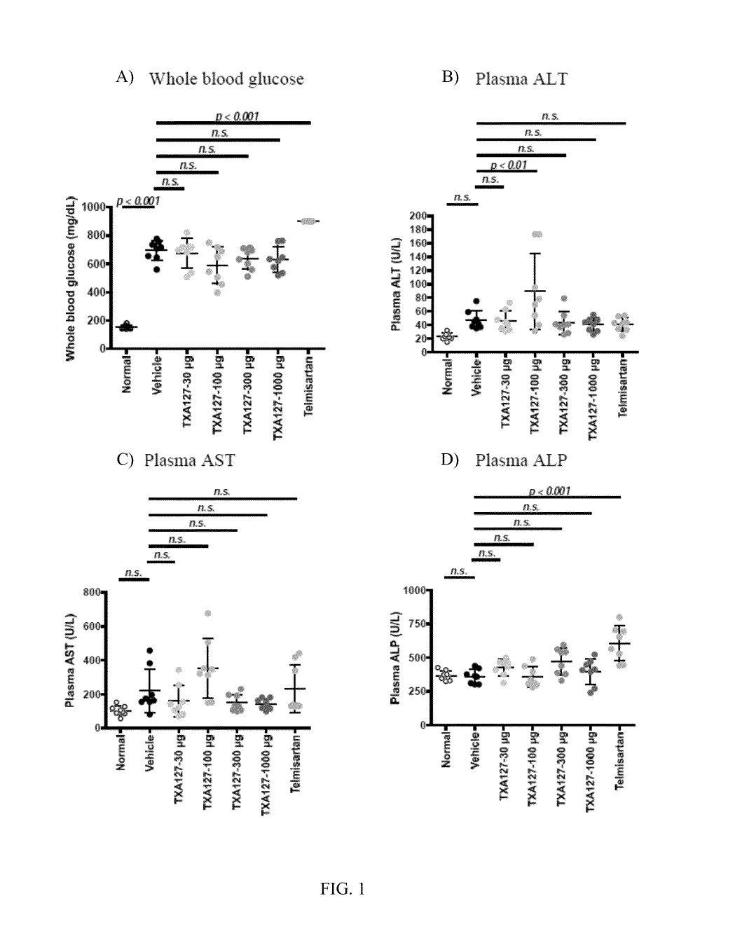

As shown in FIG. 1A, non-fasting blood glucose levels in whole blood were significantly increased in the Vehicle group compared with the Normal group (Normal: 154±15 mg/dL, Vehicle: 695±71 mg/dL). The Telmisartan group showed a significant increase in blood glucose levels compared with the Vehicle group (Telmisartan: 900±0 mg/dL). All samples in the Telmisartan group were above the detection limit of 900 mg/dL. Blood glucose levels tended to decrease in TXA127-100 μg, TXA127-300 μg, and TXA127-1000 μg groups compared with the Vehicle group (TXA127-100 μg: 590±131 mg/dL, TXA127-300 μg: 639±76 mg/dL, TXA127-1000 μg: 632±92 mg/dL). There was no significant difference in blood glucose levels between the Vehicle group and TXA127-30 μg group (TXA127-30 μg: 675±103 mg/dL).

Plasma Alanine Transaminase (ALT) Results

As shown in FIG. 1B, the plasma ALT levels of the Vehicle group tended to increase compared with the Normal group (Normal: 23±5 U/L, Vehicle: 47±14 U/L). There was no significant difference in the ALT levels between the Vehicle group and the Telmisartan group (Telmisartan: 41±10 U/L). The TXA127-100 μg group showed a significant increase in the ALT levels compared with the Vehicle group (TXA127-100 μg: 89±56 U/L). There was no significant difference in the ALT levels between the Vehicle group and any of the other groups (TXA127-30 μg: 46±15 U/L, TXA127-300 μg: 43±17 U/L, TXA127-1000 μg: 41±10 U/L).

Plasma Aspartate Transaminase (AST) Results

As shown in FIG. 1C, the plasma AST levels of the Vehicle group tended to increase compared with the Normal group (Normal: 103±29 U/L, Vehicle: 220±129 U/L). There was no significant difference in the AST levels between the Vehicle group and the Telmisartan group (Telmisartan: 232±141 U/L). The AST levels of the TXA127-100 μg group tended to increase compared with the Vehicle group (TXA127-100 μg: 352±174 U/L). There was no significant difference in the AST levels between the Vehicle group and any of the other groups (TXA127-30 μg: 160±94 U/L, TXA127-300 μg: 149±50 U/L, TXA127-1000 μg: 142±32 U/L).

Plasma Alkaline Phosphatase (ALP) Results

As shown in FIG. 1D, there was no significant difference in the plasma ALP levels between the Vehicle group and the Normal group (Normal: 368±36 U/L, Vehicle: 360±53 U/L). The Telmisartan group showed a significant increase in the ALP levels compared with the Vehicle group (Telmisartan: 605±130 U/L). The ALP levels of the TXA127-30 μg and TXA127-300 μg groups tended to increase compared with the Vehicle group (TXA127-30 μg: 428±61 U/L, TXA127-300 μg: 472±100 U/L). There was no significant difference in the ALP levels between the Vehicle group and any of the other groups (TXA127-100 μg: 358±75 U/L, TXA127-1000 μg: 397±96 U/L).

Liver Hydroxyproline Results

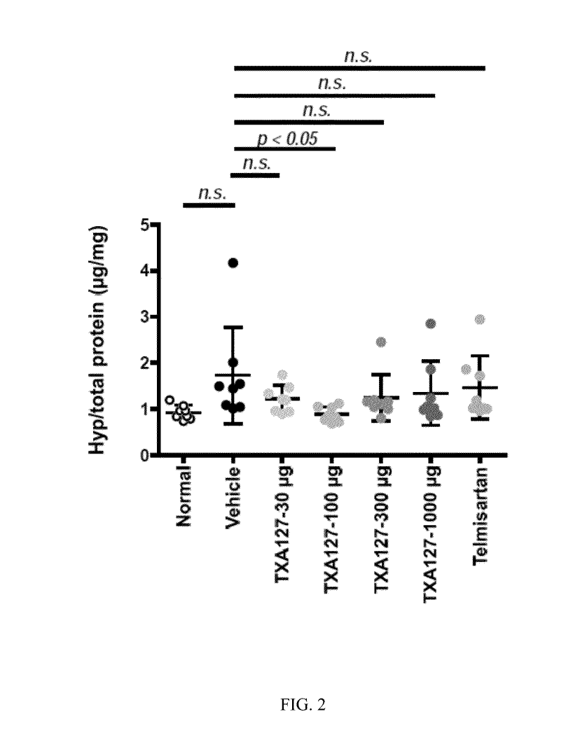

Liver hydroxyproline levels have been shown to be correlated with hepatic fibrosis and it is found specifically in collagen. As shown in FIG. 2, the liver hydroxyproline content of the Vehicle group tended to increase compared with the Normal group (Normal: 0.92±0.16 μg/mg, Vehicle: 1.72±1.04 μg/mg). There was no significant difference in the hydroxyproline content between the Vehicle group and the Telmisartan group (Telmisartan: 1.46±0.69 μg/mg). The TXA127-100 μg group showed a significant decrease in the hydroxyproline content compared with the Vehicle group (TXA127-100 μg: 0.88±0.17 μg/mg). There was no significant difference in hydroxyproline content between the Vehicle group and any of the other groups (TXA127-30 μg: 1.22±0.29 μg/mg, TXA127-300 μg: 1.24±0.51 μg/mg, TXA127-1000 μg: 1.34±0.69 μg/mg).

Sirius Red Results

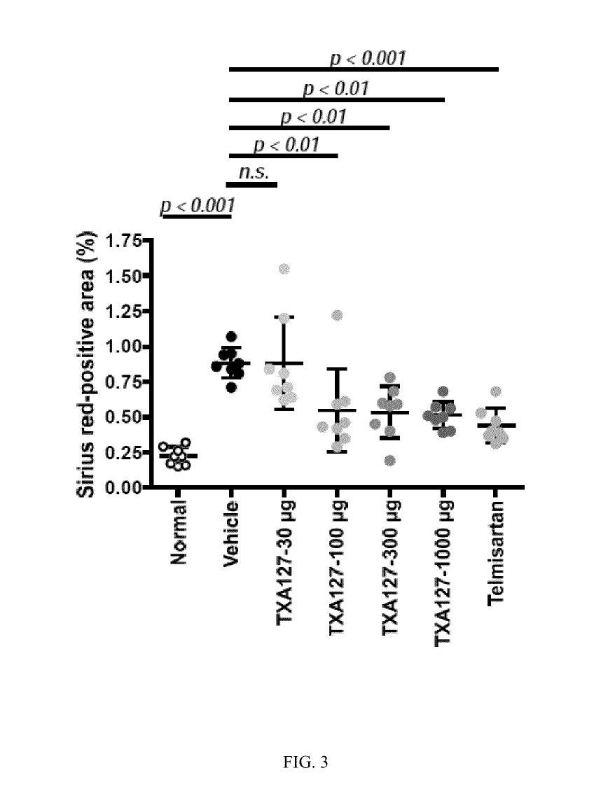

As shown in FIG. 3, Sirius red-stained liver sections of the Vehicle group showed increased collagen deposition in the pericentral region of the liver lobule compared with the Normal group. The percentage of fibrosis area (Sirius red-positive area) significantly increased in the Vehicle group compared with the Normal group (Normal: 0.22±0.06%, Vehicle: 0.88±1.10%). The fibrosis area significantly decreased in the Telmisartan group compared with the Vehicle group (Telmisartan: 0.44±0.12%). The fibrosis area significantly decreased in the TXA127-100 μg, TXA127-300 μg and TXA127-1000 μg groups compared with the Vehicle group (TXA127-100 μg: 0.55±0.29%, TXA127-300 μg: 0.54±0.18%, TXA127-1000 μg: 0.51±0.09%). There was no significant difference in the percentages of Sirius red-positive area between the Vehicle group and the TXA127-30 μg group (TXA127-30 μg: 0.88±0.33%).

Summary—Telmisartan

Telmisartan, known to show anti-inflammatory and anti-fibrosis effects in this NASH model, was used as a positive control in this study. Treatment with Telmisartan significantly decreased liver weight and NAS and the fibrosis area compared with the Vehicle group in agreement with Stelic's historical data.

Summary—TXA127

Sirus red staining revealed that treatment with TXA127 at the doses of 100, 300 and 1000 μg/kg significantly decreased collagen deposition in the pericentral region in a dose-dependent manner (see FIG. 3). On the other hand, in the hydroxyproline content, a significant decrease was observed in treatment with TXA127 at the dose of 100 μg/kg. In addition, treatment with TXA127 at all doses decreased inflammatory cell infiltration. Treatment with TXA127 at the dose of 100 μg/kg increased ALT and AST levels and at the doses of 30 and 300 μg/kg increased ALP levels. Taken together, TXA127 showed potential anti-inflammatory effects at doses above 30 μg/kg and anti-fibrosis effects at doses above 100 μg/kg in this study.

### Example 4

### Genetic Analysis of Treatment of Non-Alcoholic Steatohepatitis (NASH)

The animals, groups, treatment conditions and time points are the same as for Example 3 above. Livers were harvested from the animals in Example 3 and subjected to the following analysis.

Quantitative RT-PCR

Total RNA was extracted from liver samples using RNAiso (Takara Bio, Japan) according to the manufacturer's instructions. One μg of RNA was reverse-transcribed using a reaction mixture containing 4.5 mM MgCl2 (Roche, Switzerland), 40 U RNase inhibitor (Toyobo, Japan), 0.5 mM dNTP (Promega, USA), 6.28 μM random hexamer (Promega), 5× first strand buffer (Promega), 6.6 mM dithiothreitol (Invitrogen, USA) and MMLV-RT (Invitrogen) in a final volume of 20 μL. The reaction was carried out for 1 hour at 37° C., followed by 5 minutes at 99° C. Real-time PCR was performed using real-time PCR DICE and SYBR premix Taq (Takara Bio). To calculate the relative mRNA expression level, the expression of each gene was normalized to that of reference gene 36B4 (gene symbol: Rplp0). Statistical analyses were performed using Bonferroni Multiple Comparison Test on Prism Software 4. P values<0.05 were considered statistically significant. The expression level of Collagen Type I, collagen type 3, α-SMA, TGF-β, CCR2, and TIMP-1 mRNA were assessed.

Expression of Collagen Type I mRNA

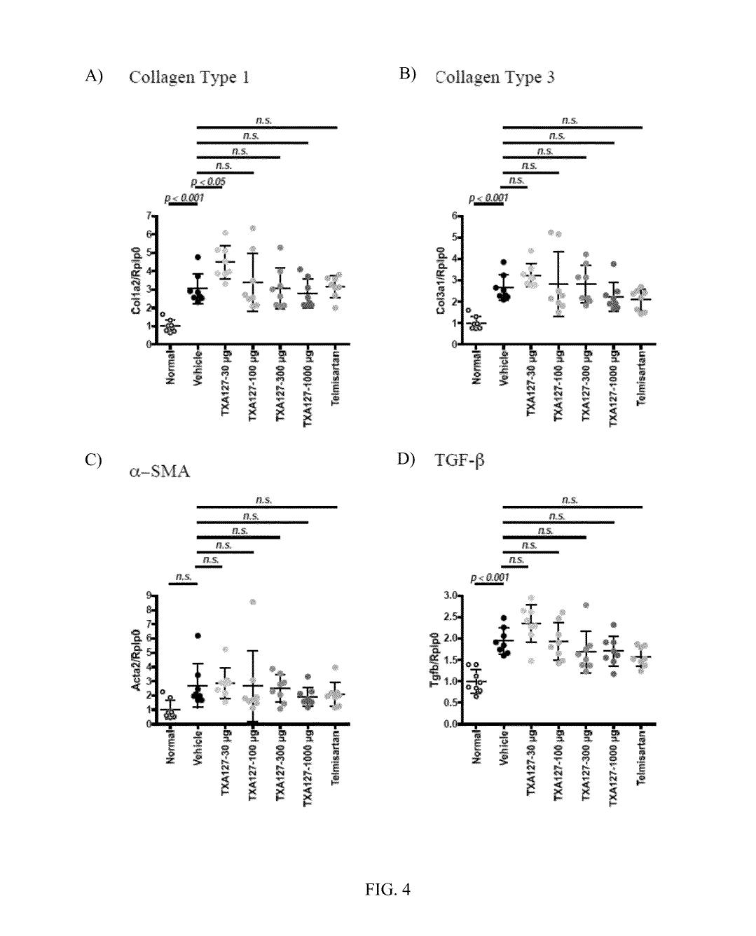

As shown in FIG. 4A, Collagen Type 1 mRNA expression levels were significantly up-regulated in the Vehicle group compared with the Normal group (Normal: 1.00±0.34, Vehicle: 3.03±0.82). There were no significant differences in Collagen Type 1 mRNA expression levels between the Vehicle group and the Telmisartan group (Telmisartan: 3.14±0.59). Collagen Type 1 mRNA expression levels were significantly up-regulated in the TXA127-30 μg group compared with the Vehicle group (TXA127-30 μg: 4.48±0.91). There were no significant differences in Collagen Type 1 mRNA expression levels between the Vehicle group and any of the other groups (TXA127-100 μg: 3.37±1.58, TXA127-300 μg: 3.06±1.12, TXA127-1000 μg: 2.77±0.77).

Expression of Collagen Type 3 mRNA

As shown in FIG. 4B, Collagen Type 3 mRNA expression levels were significantly up-regulated in the Vehicle group compared with the Normal group (Normal: 1.00±0.30, Vehicle: 2.64±0.60). Collagen Type 3 mRNA expression levels tended to be down-regulated in the Telmisartan group compared with the Vehicle group (Telmisartan: 2.09±0.50). Collagen Type 3 mRNA expression levels tended to be up-regulated in the TXA127-30 μg group compared with the Vehicle group (TXA127-30 μg: 3.23±0.54). Collagen Type 3 mRNA expression levels tended to be down-regulated in the TXA127-1000 μg group compared with the Vehicle group (TXA127-1000 μg: 2.24±0.68). There were no significant differences in Collagen Type 3 mRNA expression levels between the Vehicle group and any of the other groups (TXA127-100 μg: 2.82±1.50, TXA127-300 μg: 2.81±0.89).

Expression of α-SMA mRNA

As shown in FIG. 4C, α-SMA mRNA expression levels tended to be up-regulated in the Vehicle group compared with the Normal group (Normal: 1.00±0.67, Vehicle: 2.69±1.53). α-SMA mRNA expression levels tended to be down-regulated in the Telmisartan group compared with the Vehicle group (Telmisartan: 2.07±0.87). α-SMA mRNA expression levels tended to be down-regulated in the TXA127-1000 μg group compared with the Vehicle group (TXA127-1000 μg: 1.92±0.67). There were no significant differences in α-SMA mRNA expression levels between the Vehicle group and any of the other groups (TXA127-30 μg: 2.88±1.08, TXA127-100 μg: 2.65±2.46, TXA127-300 μg: 2.49±0.98).

Expression of TGF-β mRNA

As shown in FIG. 4D, TGF-β mRNA expression levels were significantly up-regulated in the Vehicle group compared with the Normal group (Normal: 1.00±0.28, Vehicle: 1.94±0.31). TGF-β mRNA expression levels tended to be down-regulated in the Telmisartan group compared with the Vehicle group (Telmisartan: 1.58±0.23). TGF-β mRNA expression levels tended to be up-regulated in the TXA127-30 μg groups compared with the Vehicle group (TXA127-30 μg: 2.35±0.44). TGF-β mRNA expression levels tended to be down-regulated in the TXA127-300 μg and TXA127-1000 μg groups compared with the Vehicle group (TXA127-300 μg: 1.69±0.49, TXA127-1000 μg: 1.70±0.35). There were no significant differences in a-SMA mRNA expression levels between the Vehicle group and the TXA127-100 μg group (TXA127-100 μg: 1.93±0.44).

Expression of CCR2 mRNA

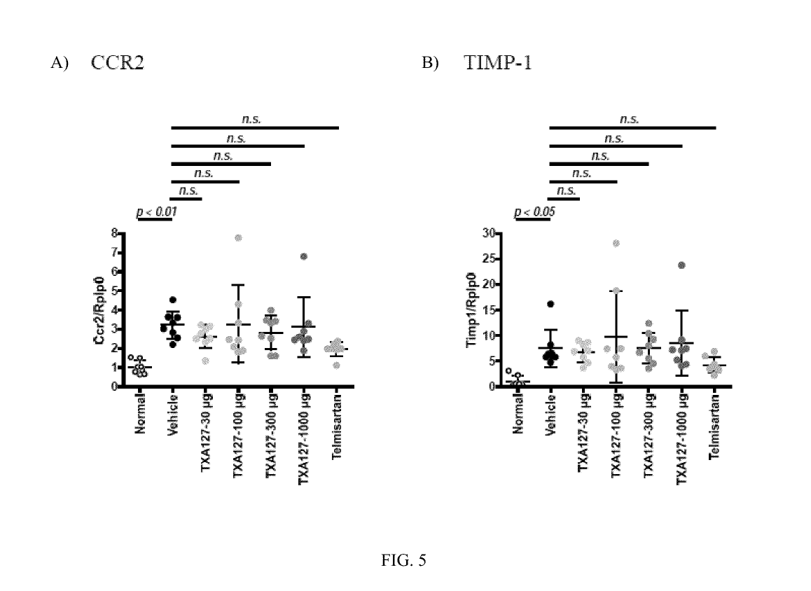

As shown in FIG. 5A, CCR2 mRNA expression levels were significantly up-regulated in the Vehicle group compared with the Normal group (Normal: 1.00±0.35, Vehicle: 3.22±0.73). CCR2 mRNA expression levels tended to be down-regulated in the Telmisartan group compared with the Vehicle group (Telmisartan: 1.94±0.37). CCR2 mRNA expression levels tended to be down-regulated in the TXA127-30 μg group compared with the Vehicle group (TXA127-30 μg: 2.60±0.60). There were no significant differences in CCR2 mRNA expression levels between the Vehicle group and any of the other groups (TXA127-100 μg: 3.26±2.01, TXA127-300 μg: 2.82±0.88, TXA127-1000 μg: 3.10±1.55).

Expression of TIMP-1 mRNA

As shown in FIG. 5B, TIMP-1 mRNA expression levels were significantly up-regulated in the Vehicle group compared with the Normal group (Normal: 1.00±1.07, Vehicle: 7.46±3.66). TIMP-1 mRNA expression levels tended to be down-regulated in the Telmisartan group compared with the Vehicle group (Telmisartan: 4.22±1.52). There were no significant differences in TIMP-1 mRNA expression levels between the Vehicle group and any of the other groups (TXA127-30 μg: 6.74±1.93, TXA127-100 μg: 9.80±8.93, TXA127-300 μg: 7.54±3.05, TXA127-1000 μg: 8.54±6.41).

Summary

In this study, treatment with Telmisartan appeared to down-regulate the expression levels of Collagen Type 3, α-SMA, TGF-13, CCR2 and TIMP-1 mRNA. Since treatment with Telmisartan significantly decreased fibrosis area in Example 3, these results support the anti-fibrosis effect of Telmisartan and its use as a positive control.

TXA127 showed anti-fibrosis and anti-inflammatory effects in Example 3. In this study, treatment with TXA127 reduced the TGF-β gene expression levels in a dose-dependent manner, and treatment with TXA127 at the dose of 1,000 μg/kg tended to down-regulate the Collagen Type 3 and α-SMA mRNA expression levels. Without wishing to be held to a particular theory, these results may indicate that TXA127 ameliorates fibrosis through suppression of activation of hepatic stellate cells induced by TGF-β. It has been reported that the angiotensin-(1-7) peptide suppresses activation of macrophage (TGF-β producing cells) and hepatic stellate cells via mas receptor. One possible mechanism of action is that TXA127 reduces activation of macrophages and the number of TGF-β-stimulated α-SMA positive cells, leading to reduced fibrosis.

### Example 5

### Treatment of Cystic Fibrosis with Linear or Cyclic A(1-7)

A chronic airway infection model mimicking cystic fibrosis is established by intratracheal instillation of a mucoid strain of Pseudomonas aeruginosa (NH57388A) into the airways of 10-12 week old BALB/c mice (Charles River). NH57388A is a mucA knockout mutant that overproduces alginate which confers resistance to host immunity. Ang (1-7) and cyclic A(1-7) are administered using two pharmaceutical preparations. After 24 hours of infection the animals receive treatment with either linear Ang (1-7) (100 or 300 mcg/kg) or cyclic Ang (1-7) (10 or 30 mcg/kg) via an implantable pump (alzet) for 7 days. Treated mice and controls are euthanized by i.p. injection of 20 mg sodium pentobarbital on day 8. Bronchoalveolar lavage (BAL) is performed by cannulating the trachea and lavaging with 0.8 mL sterile saline 3 times. The supernatant is aliquoted and stored at −70° C. for further biochemical measurements. Total and differential cell counts are performed on cytospin preparations using DIFFQUICK™ staining Histopathology is performed on lung tissue to determine the extent of lung injury. Hematoxylin and eosin (H&E) staining is performed to examine neutrophil infiltration into the lung tissue. Inflammatory biomarker concentrations (e.g. IL1β, KC, MIP2, IFNγ, TNFα, IL-6, MCP-1, IL-10) in BAL fluid is determined using multiplex ELISAs. Mas mRNA is analyzed by qRT-PCR performed on lung tissue.

A sample size of 8 animals per group provides a 90% chance of detecting a 2 log drop in neutrophil counts within BAL between Ang (1-7) treated and control animals with 95% confidence. Neutrophil counts and cytokine/chemokine concentrations are analyzed by the Mann-Whitney U test. The null hypothesis is rejected at p<0.05. Statistical analyses are performed using GRAPHPAD™ Prism for Mac version 5.0b (GRAPHPAD™, San Diego, Calif., USA). These preliminary studies will demonstrate the benefits of CF treatment CF by A(1-7). For example, the magnitude and duration of anti-inflammatory dose response elicited by A(1-7) will be demonstrated.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. The articles “a”, “an”, and “the” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses variations, combinations, and permutations in which one or more limitations, elements, clauses, descriptive terms, etc., from one or more of the claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth herein. It should also be understood that any embodiment of the invention, e.g., any embodiment found within the prior art, can be explicitly excluded from the claims, regardless of whether the specific exclusion is recited in the specification.

It should also be understood that, unless clearly indicated to the contrary, in any methods claimed herein that include more than one act, the order of the acts of the method is not necessarily limited to the order in which the acts of the method are recited, but the invention includes embodiments in which the order is so limited. Furthermore, where the claims recite a composition, the invention encompasses methods of using the composition and methods of making the composition. Where the claims recite a composition, it should be understood that the invention encompasses methods of using the composition and methods of making the composition.

### INCORPORATION OF REFERENCES

All publications and patent documents cited in this application are incorporated by reference in their entirety to the same extent as if the contents of each individual publication or patent document were incorporated herein.

1. A method of treating or preventing a fibrotic disease, disorder or condition, the method comprising administering to a subject in need of treatment Angiotensin (1-7) or an analog or derivative thereof.

2. The method of claim 1, wherein the fibrotic disease, disorder or condition comprises lung fibrosis.

3. The method of claim 2, wherein the lung fibrosis is selected from the group consisting of pulmonary fibrosis, pulmonary hypertension, chronic obstructive pulmonary disease (COPD), asthma, cystic fibrosis, and combination thereof.

4. The method of claim 3, wherein the lung fibrosis is cystic fibrosis.

5. The method of claim 1, wherein the fibrotic disease, disorder or condition comprises kidney fibrosis.

6. The method of claim 1, wherein the fibrotic disease, disorder or condition comprises liver fibrosis.

7. The method of claim 6, wherein the liver fibrosis is non-alcoholic steatohepatitis.

8. The method of claim 1, wherein the fibrotic disease, disorder or condition comprises heart fibrosis.

9. The method of claim 1, wherein the fibrotic disease, disorder or condition is systemic sclerosis.

10. The method of claim 1, wherein the fibrotic disease, disorder or condition is caused by post-surgical adhesion formation.

11. The method of claim 1, wherein the Angiotensin (1-7) or an analog or derivative thereof is administered at a therapeutically effective amount such that at least one symptom or feature of the fibrotic disease, disorder or condition is reduced in intensity, severity, or frequency, or has delayed onset.

12.-13. (canceled)

14. The method of claim 1, wherein the method reduces or prevents scar formation on skin.

15. The method of claim 1, wherein the Angiotensin (1-7) or an analog or derivative thereof is Angiotensin (1-7) with amino acid sequence of Asp¹-Arg²-Val³-Tyr⁴-Ile⁵-His⁶-Pro⁷(SEQ ID NO:1).

16. (canceled)

17. The method of claim 1, wherein the Angiotensin (1-7) or an analog or derivative thereof has amino acid sequence of Asp¹-Arg²-Val³-Ser⁴-Ile⁵-His⁶-Cys⁷(SEQ ID NO: 3).

18. The method of claim 1, wherein the Angiotensin (1-7) or an analog or derivative thereof is a cyclic Angiotensin (1-7) polypeptide.

19. The method of claim 18, wherein the cyclic Angiotensin (1-7) polypeptide is a 4,7-cyclised Angiotensin (1-7) with the following formula:

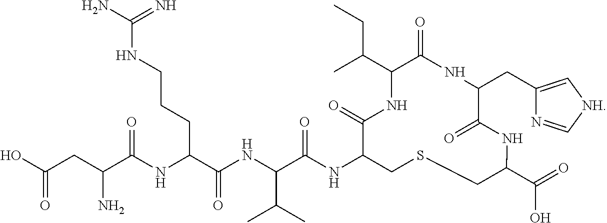 [SMILES: CCC(C)C1NC(=O)C(NC(=O)C(NC(=O)C(CCCNC(=N)N)NC(=O)C(N)CC(=O)O)C(C)C)CSCC(C(=O)O)NC(=O)C(Cc2c[nH]cn2)NC1=O]

20.-22. (canceled)

23. The method of claim 1, wherein the Angiotensin (1-7) or an analog or derivative thereof is administered parenterally.

24. The method of claim 23, wherein the parenteral administration is selected from intravenous, intradermal, inhalation, transdermal (topical), subcutaneous, and/or transmucosal administration.

25. The method of claim 1, wherein the Angiotensin (1-7) or an analog or derivative thereof is administered orally.

26. The method of claim 1, wherein the Angiotensin (1-7) or an analog or derivative thereof is administered bimonthly, monthly, triweekly, biweekly, weekly, daily, or at variable intervals.


SEQUENCE LISTING (24 sequences)

SEQ ID NO: 1 - protein, 7 aa, Artificial Sequence
  Description of Artificial Sequence Synthetic peptide
DRVYIHP

SEQ ID NO: 2 - protein, 7 aa, Artificial Sequence
  Description of Artificial Sequence Synthetic peptide
DRXYIHP

SEQ ID NO: 3 - protein, 7 aa, Artificial Sequence
  Description of Artificial Sequence Synthetic peptide
DRVSIHC

SEQ ID NO: 4 - protein, 12 aa, Artificial Sequence
  Description of Artificial Sequence Synthetic peptide
DRVYIHPFHLVI

SEQ ID NO: 5 - protein, 7 aa, Artificial Sequence
  Description of Artificial Sequence Synthetic peptide
XXXXXXX

SEQ ID NO: 6 - protein, 7 aa, Artificial Sequence
  Description of Artificial Sequence Synthetic peptide
DRVSIHC

SEQ ID NO: 7 - protein, 7 aa, Artificial Sequence
  Description of Artificial Sequence Synthetic peptide
DRVXIHX

SEQ ID NO: 8 - protein, 10 aa, Artificial Sequence
  Description of Artificial Sequence Synthetic peptide
DRXXIHXFHL

SEQ ID NO: 9 - protein, 8 aa, Artificial Sequence
  Description of Artificial Sequence Synthetic peptide
DRXXIHXF

SEQ ID NO: 10 - protein, 7 aa, Artificial Sequence
  Description of Artificial Sequence Synthetic peptide
RXXIHXF

SEQ ID NO: 11 - protein, 6 aa, Artificial Sequence
  Description of Artificial Sequence Synthetic peptide
XXIHXF

SEQ ID NO: 12 - protein, 7 aa, Artificial Sequence
  Description of Artificial Sequence Synthetic peptide
DRXXIHX

SEQ ID NO: 13 - protein, 9 aa, Artificial Sequence
  Description of Artificial Sequence Synthetic peptide
DRXXIHXFH

SEQ ID NO: 14 - protein, 7 aa, Artificial Sequence
  Description of Artificial Sequence Synthetic peptide
XXXXXHX

SEQ ID NO: 15 - protein, 7 aa, Artificial Sequence
  Description of Artificial Sequence Synthetic peptide
DRVXIHA

SEQ ID NO: 16 - protein, 7 aa, Artificial Sequence
  Description of Artificial Sequence Synthetic peptide
DRVAIHA

SEQ ID NO: 17 - protein, 10 aa, Artificial Sequence
  Description of Artificial Sequence Synthetic peptide
XXXXXHXXHX

SEQ ID NO: 18 - protein, 7 aa, Artificial Sequence
  Description of Artificial Sequence Synthetic peptide
DRXXIHA

SEQ ID NO: 19 - protein, 7 aa, Artificial Sequence
  Description of Artificial Sequence Synthetic peptide
DRXAIHA

SEQ ID NO: 20 - protein, 8 aa, Artificial Sequence
  Description of Artificial Sequence Synthetic peptide
DRXXIHAI

SEQ ID NO: 21 - protein, 8 aa, Artificial Sequence
  Description of Artificial Sequence Synthetic peptide
DRXAIHAI

SEQ ID NO: 22 - protein, 8 aa, Artificial Sequence
  Description of Artificial Sequence Synthetic peptide
DRXXIHAI

SEQ ID NO: 23 - protein, 5 aa, Artificial Sequence
  Description of Artificial Sequence Synthetic peptide
DRVYI

SEQ ID NO: 24 - protein, 6 aa, Artificial Sequence
  Description of Artificial Sequence Synthetic peptide
DRVYIH